(19) United States
(12) Patent Application Publication
(10) Pub. No.: US 2024/0225479 A1
(43) Pub. Date: Jul. 11, 2024

(54) INHALER SYSTEM

(71) Applicant: Norton (Waterford) Limited, Waterford (IE)

(72) Inventors: Guilherme Safioti, Stigtomta (SE); Mark Milton-Edwards, Cheshire (GB); Michael Reich, Herzliya (IL)

(73) Assignee: Norton (Waterford) Limited, Waterford (IE)

(21) Appl. No.: 18/558,902

(22) PCT Filed: Apr. 29, 2022
(86) PCT No.: PCT/EP2022/061541

§ 371 Date: Nov. 3, 2023

(30) Foreign Application Priority Data
May 3, 2021 (GB) 2106312.8

(51) Int. Cl. A61B 5/08; A61M 15/00; G16H 50/20

(52) U.S. Cl. CPC A61B 5/082; A61M 15/0001; G16H 50/20; A61M 2205/3334

(57) ABSTRACT

Provided is a method for generating an assessment of a respiratory disease in a subject at a current point in time. The method comprises determining a baseline statistic relating to usage of an inhaler in a baseline period. The inhaler is configured to deliver a rescue medicament to the subject, and has a use determination system configured to determine usage of the inhaler by the subject. The method also comprises determining a current statistic relating to usage of the inhaler in a current period containing the current point in time. The method further comprises generating a comparator variable. Generating the comparator variable comprises comparing the current statistic and the baseline statistic. The assessment of the respiratory disease is based on the comparator variable.

### FIELD OF THE INVENTION

This disclosure relates to an inhaler system, and particularly systems and methods for generating an assessment of a subject's respiratory disease.

### BACKGROUND OF THE INVENTION

Many respiratory diseases, such as asthma or chronic obstructive pulmonary disease (COPD), are life-long conditions where treatment involves the long-term administration of medicaments to manage the patients' symptoms and to decrease the risks of irreversible changes. There is currently no cure for diseases like asthma and COPD. Treatment takes two forms. First, a maintenance aspect of the treatment is intended to reduce airway inflammation and, consequently, control symptoms in the future.

The maintenance therapy is typically provided by inhaled corticosteroids, alone or in combination with long-acting bronchodilators and/or muscarinic antagonists. Secondly, there is also a rescue (or reliever) aspect of the therapy, where patients are given rapid-acting bronchodilators to relieve acute episodes of wheezing, coughing, chest tightness and shortness of breath. Patients suffering from a respiratory disease, such as asthma or COPD may also experience episodic flare-ups, or exacerbations, in their respiratory disease, where symptoms rapidly worsen. In the worst case, exacerbations may be life-threatening.

The ability to identify an impending respiratory disease exacerbation would improve action plans and provide opportunities for pre-emptive treatment, before the patient's condition requires, for example, unscheduled visits to or from a medical practitioner, hospital admission and administering of systemic steroids.

There is therefore a need in the art for improved methods for assessing the respiratory disease of a patient.

### SUMMARY OF THE INVENTION

Accordingly, the present disclosure provides a method for generating an assessment of a respiratory disease in a subject at a current point in time.

An exemplary method comprises determining a baseline statistic relating to usage of an inhaler in a baseline period. The inhaler is configured to deliver a rescue medicament to the subject, and has a use determination system configured to determine usage of the inhaler by the subject. The method also comprises determining a current statistic relating to usage of the inhaler in a current period containing the current point in time.

The exemplary method further comprises generating a comparator variable. Generating the comparator variable comprises comparing the current statistic and the baseline statistic. The assessment of the respiratory disease is based on the comparator variable.

In this example, the method comprises applying the comparator variable as an input to a trained machine learning model. In such embodiments, the assessment of the respiratory disease in the subject is generated as an output of the machine learning model.

An intervening period may separate the current period from the baseline period. In such embodiments, the current period and the baseline period may be regarded as being non-contiguous. The thus defined separation between the current period and the baseline period may assist the comparator variable to act as a more clear signal of any deviation from the baseline, e.g. relative to the scenario in which such periods are contiguous or overlapping.

The comparator variable, in combination with the intervening period, may thus provide a particularly useful input upon which the subject's respiratory disease can be assessed.

### BRIEF DESCRIPTION OF THE DRAWINGS

The present invention will now be described in more detail with reference to the accompanying drawings, which are not intended to be limiting:

FIG. 1 shows a block diagram of a system according to an example;

FIG. 2 shows a system according to another example;

FIG. 3 shows a method for generating a respiratory disease assessment according to a first example;

FIG. 4 shows a method for generating a respiratory disease assessment according to a second example;

FIG. 5 shows a method for generating a respiratory disease assessment according to a third example;

FIG. 6 shows a method for generating a respiratory disease assessment according to a fourth example;

FIG. 7 shows a method for generating a respiratory disease assessment according to a fifth example;

FIG. 8 shows a method for generating a respiratory disease assessment according to a sixth example;

FIG. 9 shows a method for generating a respiratory disease assessment according to a seventh example;

FIG. 10 shows a method for generating a respiratory disease assessment according to an eighth example;

FIG. 11 shows a method for generating a respiratory disease assessment according to a ninth example;

FIG. 12 shows a method for generating a respiratory disease assessment according to a tenth example;

FIG. 13 shows a method for generating a respiratory disease assessment according to an eleventh example;

FIG. 14 shows a method for generating a respiratory disease assessment according to a twelfth example;

FIG. 15 shows a method for generating a respiratory disease assessment according to a thirteenth example;

FIG. 16 shows a method for generating a respiratory disease assessment according to a fourteenth example;

FIG. 17 shows a method for generating a respiratory disease assessment according to a fifteenth example;

FIG. 18 shows a method for generating a respiratory disease assessment according to a sixteenth example;

FIG. 19 shows a method for generating a respiratory disease assessment according to a seventeenth example;

FIG. 20 shows a method for generating a respiratory disease assessment according to an eighteenth example;

FIG. 21 shows a method for generating a respiratory disease assessment according to a nineteenth example;

FIG. 22 shows a method for generating a respiratory disease assessment according to a twentieth example;

FIG. 23 shows a method for generating a respiratory disease assessment according to a twenty first example;

FIG. 24 shows a method for generating a respiratory disease assessment according to a twenty second example;

FIG. 25 shows a method for generating a respiratory disease assessment according to a twenty third example;

FIG. 26 shows a method for generating a respiratory disease assessment according to a twenty fourth example;

FIG. 27 shows a method for generating a respiratory disease assessment according to a twenty fifth example;

FIG. 28 schematically depicts a machine learning model-comprising process according to an example;

FIG. 29 schematically depicts a machine learning model-comprising process according to another example;

FIG. 30 shows a method for training a machine learning model according to an example;

FIG. 31 shows a method for training a machine learning model according to another example;

FIGS. 32 to 38 show a graphs of number of inhalations versus time during use of an inhaler for different subjects;

FIG. 39 provides a chart showing the rescue inhaler usage of a group of subjects;

FIG. 40 shows a front perspective view of an inhaler;

FIG. 41 shows a cross-sectional interior perspective view of the inhaler shown in FIG. 40;

FIG. 42 provides an exploded perspective view of the example inhaler shown in FIG. 40;

FIG. 43 provides an exploded perspective view of a top cap and electronics module of the inhaler shown in FIG. 40; and

FIG. 44 shows a graph of airflow rate through the example inhaler shown in FIG. 40 versus pressure.

### DETAILED DESCRIPTION OF THE INVENTION

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the figures to indicate the same or similar parts.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath.

The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate), fluticasone (propionate or furoate), mometasone (furoate), ciclesonide and dexamethasone (sodium). Parentheses indicate preferred salt or ester forms. Particular mention should be made of budesonide, beclomethasone and fluticasone, especially budesonide, beclomethasone dipropionate, fluticasone propionate and fluticasone furoate.

Different classes of bronchodilators target different receptors in the airways. Two commonly used classes are β₂-agonists and anticholinergics.

β₂-Adrenergic agonists (or “β₂-agonists”) act upon the β₂-adrenoceptors which induces smooth muscle relaxation, resulting in dilation of the bronchial passages. They tend to be categorised by duration of action. Examples of long-acting β₂-agonists (LABAS) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β₂-agonists (SABA) are albuterol (sulfate) and terbutaline (sulfate). Particular mention should be made of formoterol, salmeterol, indacaterol and vilanterol, especially formoterol fumarate, salmeterol xinafoate, indacaterol maleate and vilanterol triphenylacetate.

Typically short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M₃ muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). Particular mention should be made of tiotropium, aclidinium, umeclidinium and glycopyrronium, especially tiotropium bromide, aclidinium bromide, umeclidinium bromide and glycopyrronium bromide.

A number of approaches have been taken in preparing and formulating these medicaments for delivery by inhalation, such as via a dry powder inhaler (DPI), a pressurized metered dose inhaler (pMDI) or a nebulizer.

According to the GINA (Global Initiative for Asthma) Guidelines, a step-wise approach is taken to the treatment of asthma. At step 1, which represents a mild form of asthma, the patient is given an as needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localised to the airways, lung parenchyma and pulmonary vasculature. Phenotypically, these changes reduce the healthy ability of the lungs to absorb and expel gases.

Bronchitis is characterised by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Their severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β₂-agonists and corticosteroids. The medicaments are often administered as a single therapy or as combination treatments.

Patients are categorised by the severity of their COPD using categories defined in the GOLD Guidelines (Global Initiative for Chronic Obstructive Lung Disease, Inc.). The categories are labelled A-D and the recommended first choice of treatment varies by category. Patient group A are recommended a short-acting muscarinic antagonist (SAMA) prn or a short-acting β₂-agonist (SABA) prn. Patient group B are recommended a long-acting muscarinic antagonist (LAMA) or a long-acting β₂-agonist (LABA). Patient group C are recommended an inhaled corticosteroid (ICS)+a LABA, or a LAMA. Patient group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation are repeated doses of SABA, oral corticosteroids and/or antibiotics. A severe exacerbation adds controlled flow oxygen and/or respiratory support (both of which require hospitalization).

An exacerbation within the meaning of the present disclosure includes both moderate and severe exacerbations.

Provided is a method for generating an assessment of a respiratory disease in a subject at a current point in time. The method comprises determining a baseline statistic relating to usage of an inhaler in a baseline period. The inhaler is configured to deliver a rescue medicament to the subject, and has a use determination system configured to determine usage of the inhaler by the subject. The method further comprises determining a current statistic relating to usage of the inhaler in a current period containing the current point in time.

The respiratory disease may, for instance, be asthma, COPD, or cystic fibrosis.

The rescue medicament is as defined hereinabove and is typically a SABA or a rapid-onset LABA, such as formoterol (fumarate). The rescue medicine may also be in the form of a combination product, e.g. ICS-formoterol (fumarate), typically budesonide-formoterol (fumarate). Such an approach is termed “MART” (maintenance and rescue therapy). However, the presence of a rescue medicine indicates that it is an inhaler configured to deliver a rescue medicament within the meaning of the present disclosure. It therefore covers both a rescue medicament and a combination rescue and maintenance medicament. In contrast, the further inhaler described herein below, when present, is only used for the maintenance aspect of the therapy and not for rescue purposes. The key difference is that the inhaler may be used as-needed, whereas the further inhaler is intended for use at regular, pre-defined times.

In some embodiments, the inhaler is configured to deliver a rescue medicament selected from albuterol (sulfate), formoterol (fumarate), budesonide combined with formoterol (fumarate), beclomethasone (dipropionate) combined with albuterol (sulfate), and fluticasone (propionate or furoate) combined with albuterol (sulfate).

The use determination system may, for example, comprise a sensor for detecting an inhalation of the rescue medicament performed by the subject and/or a switch configured to be actuated prior to, during, or after use of the inhaler. In this way, the use determination system enables recording of each use, or attempted use, of the inhaler.

The sensor may, for example, comprise a pressure sensor, such as an absolute or differential pressure sensor.

The inhaler may, for instance, comprise a mouthpiece through which the user performs the inhalation, and a mouthpiece cover. In such an example, the switch may be configured to be actuated when the mouthpiece cover is moved to expose the mouthpiece.

In a non-limiting example, the inhaler comprises a medicament reservoir, and a dose metering assembly configured to meter a dose of the rescue medicament from the reservoir. In this particular example, the use determination system is configured to register the metering of the dose by the dose metering assembly. Each metering is thereby indicative of the rescue inhalation performed by the subject using the inhaler.

In certain examples, the use determination system employs the sensor in combination with the switch. A signal from the sensor may be used, for example, to verify whether or not a use of the inhaler, such as a dose metering, detected via the switch is accompanied by inhalation of the rescue medicament. In such non-limiting examples, the determined usage of the inhaler used in the method may comprise, or consist of, that determined via the switch and/or that determined and verified via the switch and the sensor.

In at least some embodiments, the method comprises generating a comparator variable, comprising comparing the current statistic and the baseline statistic. The assessment of the subject's respiratory disease can then based on the comparator variable.

An intervening period may separate the current period from the baseline period. In such embodiments, the current period and the baseline period may be regarded as being non-contiguous. The thus defined separation between the current period and the baseline period may assist the comparator variable to act as a more clear signal of any deviation from the baseline, e.g. relative to the scenario in which such periods are contiguous or overlapping.

The intervening period may have a fixed duration. In some embodiments, the duration of the intervening period is 3 to 15 days, preferably about 7 days. Such a duration of the intervening period may permit the baseline statistic to remain sufficiently independent of the current statistic, whilst also assisting to ensure that the baseline statistic is influenced by usage data which is sufficiently recent in order to remain a relevant indicator of the subject's baseline/“ordinary” rescue inhaler usage.

In at least some embodiments, the method is iterated repeatedly over time. Repeatedly iterating the method over time may result in the baseline statistic updating for each successive iteration, provided that the period between successive iterations is at least as long as the increment/unit of time, for example 1 day, required to pass in order for a fresh determination of the baseline statistic to be made. Thus, the baseline statistic can be regarded as providing a dynamic baseline of rescue inhaler usage.

The baseline period may have a fixed duration. In some embodiments, the duration of the baseline period is 10 to 30 days, preferably 12 to 20 days, most preferably about 20 days. Such a baseline period may balance being sufficiently long to establish the subject's baseline/“ordinary” rescue inhaler usage whilst not being so prolonged that potentially diagnostic deviations in the baseline statistic risk becoming less pronounced.

The baseline period may be defined such as to exclude a period in which an exacerbation of the subject's respiratory disease takes place. Such an excluded period may extend from prior to the exacerbation (e.g. at a point at which the subject's condition is observed to start deteriorating prior to the exacerbation) to a point at which the subject is considered to have recovered from the exacerbation. In other words, the subject may not experience an exacerbation of their respiratory disease in the baseline period. This reflects the role of the baseline statistic in tracking the subject's baseline/“ordinary” rescue inhaler usage.

Should an exacerbation occur, the usage data for apposite periods of time before and after the exacerbation may, for example, be removed from the determination of the baseline statistic, so as not to influence the baseline statistic and correspondingly the assessment of the subject's respiratory disease.

In some embodiments, the duration of the current period is 24 hours to 120 hours, preferably about 48 hours. Such a current period may balance being sufficiently long to enable collection of a suitable amount of inhaler usage data, whilst not being so prolonged that the current statistic risks becoming less representative of the inhaler usage status at the current point in time.

More generally, it is noted that the term “current period” may refer to a time period which extends backwards in time from the current point in time into the immediate past. Sampling inhaler usage data in this time period thus enables determination of the current statistic. The current period may not extend beyond the current point in time into the future, since inhaler usage data required for determination of the current statistic is not yet available.

Generating the assessment of the subject's respiratory disease based on the comparator variable can be implemented in any suitable manner. In at least some embodiments, the comparator variable is applied as an input to a trained machine learning model.

Any suitable machine learning model can be considered, such as a supervised machine learning model. In a non-limiting example, the model is constructed using a decision trees technique. Other suitable techniques, such as building a neural network or a deep learning model may also be contemplated. Construction and training of the machine learning model are described in more detail herein below.

Any suitable baseline statistic can be considered provided that the baseline statistic is indicative of the subject's usage of the inhaler during the baseline period.

In some embodiments, the baseline statistic comprises one or more of a baseline average number of rescue inhalations using the inhaler per unit time, a baseline standard deviation of the number of rescue inhalations using the inhaler per unit time, and a baseline coefficient of variance of the number of rescue inhalations per unit time, calculated over the baseline period.

Any suitable baseline average number of rescue inhalations per unit time can be considered, such as a mean, a median, and/or a mode of the number of rescue inhalations using the inhaler per unit time calculated over the baseline period. Particular mention is made of the daily mean number of rescue inhalations during the baseline period.

It is noted that the term “coefficient of variance” as used herein may be defined by the standard deviation of the number of rescue inhalations using the inhaler per unit time divided by the mean number of rescue inhalations per unit time.

More generally, more than one input may, for example, be applied to the machine learning model. In other words, a plurality of inputs may be applied to the machine learning model, including the comparator variable. In some embodiments, the baseline statistic is itself applied as an input to the trained machine learning model.

Any suitable current statistic can be considered provided that the current statistic is indicative of the subject's usage of the inhaler during the current period.

In some embodiments, determining the current statistic comprises determining a total current number of rescue inhalations summed over the current period.

Alternatively or additionally, the current statistic comprises one or more of a current average number of rescue inhalations using the inhaler per unit time, a current standard deviation of the number of rescue inhalations using the inhaler per unit time, and a current coefficient of variance of the number of rescue inhalations per unit time, calculated over the current period.

Any suitable current average number of rescue inhalations per unit time can be considered, such as a mean, a median, and/or a mode of the number of rescue inhalations using the inhaler per unit time calculated over the current period. Particular mention is made of the daily mean number of rescue inhalations during the current period.

In some embodiments, the current statistic is itself applied as an input to the trained machine learning model (as well as the comparator variable), for example as an alternative or in addition to the baseline statistic. For example, the assessment of the subject's respiratory disease may be partly based on whether the daily mean number of rescue inhalations during the current period reaches or exceeds a defined threshold, such as ≥3.

It is noted that the term “the assessment of the subject's respiratory disease may be partly based on” as used herein may mean that the associated feature or parameter upon which the assessment is being partly based can, for example, be applied as an input in the trained machine learning model.

In some embodiments, the method comprises determining an interim statistic relating to usage of the inhaler in the intervening period.

In some embodiments, the method further comprises applying the interim statistic and/or data derived from the interim statistic as an input or inputs to the trained machine learning model. Alternatively or additionally, generating the comparator variable further comprises comparing the interim statistic with the current statistic and/or the baseline statistic. The assessment of the subject's respiratory disease may thus be additionally guided by a more recent trend in inhaler usage than provided via the baseline statistic.

Any suitable interim statistic can be considered provided that the interim statistic is indicative of the subject's usage of the inhaler during the intervening period.

In some embodiments, determining the interim statistic comprises determining a total intervening number of rescue inhalations summed over the intervening period.

In a non-limiting example, determining the interim statistic comprises comparing the total intervening number of rescue inhalations to a given threshold number of rescue inhalations in the intervening period.

For example, the assessment of the subject's respiratory disease may be partly based on the total intervening number of rescue inhalations being less than or equal to a given threshold.

For example, the assessment of the subject's respiratory disease may be partly based on an assessment of whether the total number of rescue inhalations is equal to zero in an intervening period, e.g. an intervening period having a duration of 7 days.

Alternatively or additionally, the interim statistic comprises one or more of an interim average number of rescue inhalations using the inhaler per unit time, an interim standard deviation of the number of rescue inhalations using the inhaler per unit time, and an interim coefficient of variance of the number of rescue inhalations per unit time, calculated over the intervening period.

Any suitable interim average number of rescue inhalations per unit time can be considered, such as a mean, a median, and/or a mode of the number of rescue inhalations using the inhaler per unit time calculated over the intervening period. Particular mention is made of the daily mean number of rescue inhalations during the intervening period.

In some embodiments, comparing the current statistic and the baseline statistic comprises comparing the baseline average and the current average, for example by comparing the baseline mean number of rescue inhalations per unit time calculated over the baseline period and the current mean number of rescue inhalations per unit time calculated over the current period.

The unit time used for calculation of the baseline statistic may, for instance, be the same as the unit time used for calculation of the current statistic and, where applicable the interim statistic, in order to facilitate comparison between the respective statistics.

In particular, the unit time used for calculation of the baseline average number of rescue inhalations, the baseline standard deviation of the number of rescue inhalations and/or the baseline coefficient of variance of the number of rescue inhalations is the same as the unit time used for the corresponding calculation of the current average number of rescue inhalations, the current standard deviation of the number of rescue inhalations and/or the current coefficient of variance of the number of rescue inhalations.

In embodiments in which the method is iterated repeatedly over time, consecutive iterations of the method may be separated from each other by one or more of the unit time used for calculation of the baseline statistic.

In some embodiments, comparing the current statistic and the baseline statistic comprises calculating a difference between the baseline average and the current average, for example by calculating a difference between the baseline mean and the current mean.

In a non-limiting example, comparing the current statistic and the baseline statistic comprises comparing the difference between the baseline average and the current average to a predetermined difference threshold.

The predetermined threshold can be defined in any suitable manner, such as using a particular number, or using a statistical test, such as a valid/common statistical test, e.g. using the standard deviation of the rescue inhaler usage in the baseline period.

The assessment of the subject's respiratory disease may, for example, be based on the current average being greater than the baseline average by a difference which reaches or exceeds the predetermined difference threshold.

For example, the assessment of the subject's respiratory disease may be partly based on an assessment of whether the daily mean number of rescue inhalations in the current period is at least 3 more than the daily mean number of rescue inhalations in the baseline period.

Alternatively or additionally, comparing the current statistic and the baseline statistic comprises calculating a ratio of the current average to the baseline average, for example by calculating a ratio of the daily mean number of rescue inhalations in the current period to the daily mean number of rescue inhalations in the baseline period.

In a non-limiting example, comparing the current statistic and the baseline statistic comprises comparing the ratio of the current average to the baseline average to a predetermined ratio threshold.

The predetermined ratio threshold can be defined in any suitable manner, such as using a particular number, or using a statistical test, such as a valid/common statistical test.

For example, the assessment of the subject's respiratory disease may be partly based on an assessment of whether the daily mean number of rescue inhalations in the current period is at least twice the daily mean number of rescue inhalations in the baseline period.

Generating the comparator variable may, for example, comprise both of the above-described difference and ratio determinations. This may provide certain benefits over using the difference without the ratio, or the ratio without the difference.

For a user whose daily mean rescue inhaler uses in the baseline period is relatively high, an increase in rescue inhaler use in the current period may be more evident from the difference between the current average and the baseline average than from the ratio of the current average to the baseline average. Conversely, for a user whose daily mean rescue inhaler uses in the baseline period is relatively low, an increase rescue inhaler use may be more evident from the ratio of the current average to the baseline average than from the difference. Accordingly, making use of the ratio and the difference may account for these two different types of subject.

In a non-limiting example, the assessment of the subject's respiratory disease is partly based on the assessment of whether the daily mean number of rescue inhalations in the current period is at least twice the daily mean number of rescue inhalations in the baseline period; and based on the assessment of whether the daily mean number of rescue inhalations in the current period is at least 3 more than the daily mean number of rescue inhalations in the baseline period.

An excessive rescue inhaler usage measure may be termed a “SABA burst”, and may be defined by one or more of the following: the current average reaching or exceeding a predetermined threshold; the ratio of the current average to the baseline average reaching or exceeding a predetermined ratio threshold; the current average being greater than the baseline average by a difference which reaches or exceeds a predetermined difference threshold; and on the total intervening number of rescue inhalations being less than or equal to a given threshold.

In a non-limiting example, the excessive rescue inhaler usage measure, e.g. a SABA burst, is defined by:

the daily average number of inhalations in the last 2 days (current period) being at least 3, and there is an increase in daily average number of inhalations—any one of the following:
daily average number of inhalations in last 2 days (current period) is a factor of 2 or more than the daily average number of inhalations in previous 2 weeks prior to last week (days (−8) to (−20); baseline period)
daily average number of inhalations in last 2 days (current period) is +3 or more than daily average number of inhalations in previous 2 weeks prior to last week (days (−8) to (−20); baseline period)
daily average number of inhalations in last 2 days (current period) is at least 3, and 0 inhalations in last 7 days (days (−2) to (−8); intervening period).

It is reiterated that the definition of the excessive rescue inhaler usage can include any suitable statistic test, e.g. a valid or common type of statistical test. Accordingly as an alternative or in addition to any or all of the above-defined SABA burst criterion/criteria, a rule may, for instance, be defined that if the subject has a baseline usage of X inhalations per day, e.g. a baseline mean daily rescue inhaler usage of X inhalations per day, with standard deviation S, and a SABA burst is defined if in current period the subject has more than X+1.5*S inhalations per day.

More generally, the assessment may be based on any combination of the current statistic, the baseline statistic, and/or the comparator variable.

In some embodiments, the method comprises determining a current inhalation parameter statistic from a determined parameter relating to airflow during an inhalation performed by the subject using an inhaler during the current period.

The inhaler from which the parameter relating to airflow is determined can be any suitable inhaler, such as the above-described inhaler configured to deliver the rescue medicament.

Alternatively or additionally, the parameter relating to airflow may be determined from an inhalation performed by the subject using a further inhaler configured to deliver a maintenance medicament to the subject during a routine inhalation.

The further inhaler may, for example, be configured to deliver a maintenance medicament selected from budesonide, beclomethasone (dipropionate), fluticasone (propionate or furoate), and salmeterol (xinafoate) combined with fluticasone (propionate or furoate).

A sensor system may be included in the inhaler, e.g. the inhaler and/or the further inhaler, and configured to measure the parameter relating to airflow.

Thus, the sensor system may be configured to sense the parameter during rescue inhalations of the rescue medicament performed by the subject using the inhaler and/or during routine inhalations of the maintenance medicament performed by the subject using the further inhaler.

The parameter relating to airflow during the inhalation may act as a proxy for the lung condition of the subject. Any suitable parameter relating to airflow can be considered. In a non-limiting example, the parameter comprises, or consists of, at least one of a peak inhalation flow, an inhalation volume, a time to peak inhalation flow, and an inhalation duration.

The sensor system may comprise any suitable sensor for sensing the parameter relating to airflow, such as such as an absolute or differential pressure sensor.

In examples in which the use determination system includes the sensor for detecting an inhalation (e.g. in order to verify an attempted usage event detected via the above-described switch), that sensor may be the same as or different from the sensor included in the sensor system for sensing the parameter relating to airflow.

In some embodiments, the current inhalation parameter statistic and/or data derived from the current inhalation parameter statistic may, for instance, be used to generate the assessment of the subject's respiratory disease, for example by being applied as an input or inputs to the trained machine learning model.

Alternatively or additionally, generating the comparator variable may further comprise modifying the baseline statistic, the current statistic and/or the comparison of the baseline statistic and the current statistic using the current inhalation parameter statistic.

In some embodiments, the method comprises determining a baseline inhalation parameter statistic from a determined parameter relating to airflow during an inhalation performed by the subject using an inhaler during the baseline period.

Such a baseline inhalation parameter statistic and/or data derived from the baseline inhalation parameter statistic may, for example, be applied as an input or inputs to the trained machine learning model.

In a non-limiting example, generating the comparator variable further comprises comparing the current inhalation parameter statistic and the baseline inhalation parameter statistic.

More generally, the method comprises generating, as an output of the trained machine learning model, the assessment of the respiratory disease in the subject.

Construction of the machine learning model may involve solving a classification problem. Such a classification problem may be defined based on one or more labels, in other words known values of a response variable. The values of the response variable may be, for example, yes/no to an exacerbation of the subject's respiratory disease, e.g. as diagnosed via a clinical assessment, occurring in an upcoming defined time period and/or the subject fulfilling an excessive rescue inhaler usage measure indicative of excessive rescue inhaler usage in an upcoming defined time period, etc. Further explanation of possible labels (and outputs of the machine learning model) is provided herein below.

The comparator variable, and in some embodiments the above-described current statistic, baseline statistic, interim statistic, current inhalation parameter statistic and/or baseline inhalation parameter statistic, is or are used as input(s), in other words feature(s), applied to the machine learning model. Training data used to train the machine learning model may comprise the input(s) and the label(s) for each of a plurality of training subjects.

An optimization algorithm may then use the training data to minimize a suitable loss function, which loss function may be a function of the difference between an estimated and a true value of the response variable. The optimization algorithm may use the known values of the response variable and the corresponding values of the input(s) to minimize the expected value of the loss function.

In a non-limiting example, the supervised machine learning technique, Gradient Boosting Trees, is used to construct the machine learning model. Such a Gradient Boosting Trees technique may be implemented using any suitable software. Particular mention is made of the XGBoost open-source software library.

The Gradient Boosting Trees technique is well-known in the art. See: J. H. Friedman, Computational Statistics & Data Analysis 2002, 38(4), 367-378; and J. H. Friedman et al., The Annals of Statistics 2000, 28(2), 337-407. It can produce a prediction model in the form of an ensemble (multiple learning algorithms) of base prediction models, which are decision trees (a tree-like model of decisions and their possible consequences). The above-described optimization algorithm minimizes the suitable loss function in order to build a single strong learner model in an iterative fashion. The training set of known values of the response variable (e.g. yes/no exacerbation in the upcoming time period) and the corresponding values of input(s) (e.g. including the comparator variable) are used to minimize the expected value of the loss function. The learning procedure consecutively fits new models to provide a more accurate estimate of the response variable.

In a first set of embodiments, the assessment of the respiratory disease comprises a prediction of the subject's future usage of the inhaler.

Such a prediction may be of the subject's future usage of the inhaler in a prediction period which extends forward in time from the current point in time. For example, the prediction period may be a period of time which immediately follows the current point in time. The duration of the prediction period may be, for instance, 1 to 14 days, preferably 3 to 10 days, most preferably about 5 days.

The prediction period may be selected based on the capability of the model to predict the subject's future usage of the inhaler within such a period, whilst also ensuring that the prediction period is sufficiently long for appropriate therapeutic steps to be taken, if necessary.

The prediction of the subject's future usage of the inhaler may, for example, comprise a prediction of one or more parameters relating to airflow during an inhalation performed by the subject using the inhaler.

The one or more parameters relating to airflow may comprise, or consist of, at least one of a peak inhalation flow, an inhalation volume, a time to peak inhalation flow, and an inhalation duration.

In a non-limiting example, the assessment of the respiratory disease comprises a prediction of the subject's peak inhalation flow (PIF), or in some cases peak expiratory flow (PEF and/or some other measure of expiratory flow). The parameter relating to airflow during inhalation, such as PIF, or during exhalation, such as PEF, may provide a measure of the user's lung function.

Such a measure may be impractical to obtain in certain cases, such as when the subject's inhaler does not include a suitable sensor system for determining the parameter relating to airflow. The determined usage of the inhaler may nonetheless be used to predict the parameter, and thus predict the user's lung function.

In a specific non-limiting example, values of the response variable may comprise or consist of yes/no to whether there is a decrease in the inhalation volume to or below a predetermined threshold (e.g. a 20% decrease with respect to a baseline inhalation volume); and/or yes/no to whether there is a decrease in peak inhalation flow (e.g. a 5% decrease with respect to a baseline peak inhalation flow, and the decrease is statistically significant, e.g. with reference to the standard deviation of the peak inhalation flow).

Alternatively or additionally, the prediction of the subject's future usage of the inhaler comprises a prediction of the subject later fulfilling an excessive rescue inhaler usage measure indicative of excessive rescue inhaler usage. Such an excessive rescue inhaler usage measure may, for instance, correspond the definition of a “SABA burst” provided herein.

In a second set of embodiments, which be an alternative or in addition to the above-described first set of embodiments, the assessment of the respiratory disease comprises an approximation to a clinically determined indication of the status of the subject's respiratory disease.

The term “approximation to a clinically determined indication” is distinguished from a “clinically determined indication” in that the former is an output determined by the trained machine learning model, not by a clinician. In other words, the model is trained to approximate the clinical assessment.

The assessment of the respiratory disease may comprise a prediction of the subject later suffering an exacerbation of their respiratory disease.

Such a prediction may be of the subject suffering an exacerbation of their respiratory disease in an exacerbation prediction period which extends forward in time from the current point in time. For example, the exacerbation prediction period may be a period of time which immediately follows the current point in time. The duration of the exacerbation prediction period may be, for instance, 1 to 14 days, preferably 3 to 10 days, most preferably about 5 days.

The exacerbation prediction period may be selected based on the capability of the model to predict an exacerbation within such a period, whilst also ensuring that the prediction period is sufficiently long for appropriate therapeutic steps to be taken, if necessary.

In some embodiments, the machine learning model is trained, and/or is adapted from an initial machine learning model trained, with training data comprising, for each of a plurality of training subjects, a historical comparator variable and label data.

The historical comparator variable may be generated by comparing a historical baseline statistic relating to usage of the inhaler in a historical baseline period and a subsequent statistic relating to usage of the inhaler in a subsequent period, with the subsequent period being subsequent to the historical baseline period.

The label data may, for instance, comprise a measure of each of the training subjects' usage of the inhaler determined after the subsequent period.

In such a non-limiting example, the measure of each of the training subjects' usage of the inhaler determined after the subsequent period may, for instance, comprise an assessment of whether each training subject fulfilled the above-described excessive rescue inhaler usage measure indicative of excessive rescue inhaler usage, e.g. experienced a SABA burst.

In another specific non-limiting example, values of the response variable may comprise or consist of at least one of the following:
yes/no to whether there are any rescue inhaler uses on 2 consecutive days after a 7 day period without any usage;
yes/no to whether the mean daily number of rescue inhaler uses equals or exceeds an absolute threshold (e.g. 1.5 uses per day) and equals or exceeds a threshold defined with reference to a baseline mean daily number of rescue inhaler uses (e.g. is at least a factor of 2 greater than the baseline mean daily number of rescue inhaler uses);
yes/no to whether there is an increase in rescue inhaler uses with time that can be modelled by a statistically significant linear regression, and the slope is equal to or greater than a slope threshold (e.g. 1 rescue inhalation per day); and
yes/no to whether there is a night rescue inhaler use (e.g. between 00:00 and 05:00) after there being no night rescue inhaler use for a certain number of days (e.g. 0 to 3 days).

In some embodiments, the machine learning model is adapted from the initial machine learning model with further label data comprising a clinically determined indication of the status of the respiratory disease for each of a plurality of clinical assessment subjects.

In such embodiments, the further label data may, for example, comprise an indication, for each of the plurality of clinical assessment subjects, of whether the respective clinically assessed subject suffered an exacerbation of their respiratory disease.

Examples of such a two-step machine learning model training process is described in more detail herein below.

More generally, the method may also comprise controlling a user interface to communicate a notification based on the generated assessment of the subject's respiratory disease.

The notification may, for example, comprise a warning and/or recommendation, e.g. a recommendation for the subject to seek medical attention and/or take some other pre-emptive step. Such a warning and/or recommendation may, for example, be communicated via the user interface should the generated assessment of the subject's respiratory disease be indicative of worsening of the subject's respiratory disease, such as acute worsening indicative of an impending exacerbation.

Alternatively or additionally, the notification may be in the form of a prompt for prompting the subject to provide an indication of the status of their respiratory disease.

In this manner, the inhaler usage data may be supplemented by additional status input from the subject. The user-inputted indication may provide information which confirms or validates the generated assessment.

Moreover, this approach to prompting user-inputting of the indication based on the generated assessment may reduce the burden on the subject as compared to, for example, the scenario in which the user is routinely prompted to input the indication, irrespective of their inhaler use. This, in turn, may render it more likely that the subject will input the indication when prompted to do so. Thus, improved monitoring of the subject's respiratory disease may be enabled.

Further provided is a method for treating a respiratory disease exacerbation in a subject, the method comprising: performing the method as defined above; and treating the respiratory disease based on the generated assessment.

The treatment may comprise modifying an existing treatment. The existing treatment may comprise a first treatment regimen, and the modifying the existing treatment of the respiratory disease may comprise changing from the first treatment regimen to a second treatment regimen based on the generated assessment, wherein the second treatment regimen is configured for a worse condition of the subject's respiratory disease, e.g. with a higher risk of a respiratory disease exacerbation, than the first treatment regimen.

Thus, the generated assessment may have the potential to guide intervention for a subject whose respiratory disease could be better treated and/or managed. Implementing the second treatment regimen may, for example, involve progressing the subject to a higher step specified in the GINA or GOLD guidelines. Such preemptive intervention may, for example, mean that the subject need not proceed to suffer the exacerbation, and be subjected to the associated risks, in order for the progression to the second treatment regimen to be justified.

In an embodiment, the second treatment regimen comprises administering a biologics medication to the subject. The relatively high cost of biologics means that stepping up the subject's treatment to include administering of a biologics medication tends to require careful consideration and justification. The generated assessment may provide a reliable metric to justify administering of a biologics medication.

The term “biologics medication” may refer to a medicine that contains one or more active substances made by or derived from a biological source.

The biologics medication may comprise one or more of omalizumab, mepolizumab, reslizumab, benralizumab, and dupilumab.

Modifying the existing treatment of the respiratory disease may comprise changing from the first treatment regimen to a third treatment regimen based on the generated assessment. The third treatment regimen may be configured for lower risk of a respiratory disease exacerbation than the first treatment regimen.

In this non-limiting example, the generated assessment may be used as guidance to justify downgrading or even removal of an existing treatment regimen. This may, for example, involve progressing the subject to a lower step specified in the GINA or GOLD guidelines.

Thus, the generated assessment may be used to monitor subject recovery, and may, for instance, be used to justify withdrawal of oral steroids or other medication. This may assist to lessen the risk of hospital/healthcare setting re-admission.

Further provided is a method for training a machine learning model for use in generating an assessment of a respiratory disease in a subject. The thus trained machine learning model may, for example, be utilized in the above-described method for generating the assessment of the subject's respiratory disease.

The method for training the machine learning model comprises, for each of a plurality of training subjects, determining a baseline statistic relating to usage of an inhaler in a baseline period. The inhaler is configured to deliver a rescue medicament to the training subject, and has a use determination system configured to determine usage of the inhaler by the training subject, as previously described.

The method for training the machine learning model also comprises determining, for each of the plurality of training subjects, a subsequent statistic relating to usage of the inhaler in a subsequent period.

An intervening period may separate the subsequent period from the baseline period, similarly to in the above-described embodiments of the method for generating the assessment of the subject's respiratory disease in which the baseline period is separated from the current period by the intervening period.

In this training method, a comparator variable is generated for each of the plurality of training subjects. Generating the comparator variable comprises comparing the subsequent statistic and the baseline statistic.

For each of the training subjects, label data, comprising an assessment of the respiratory disease in the respective training subject, is obtained.

The method further comprises generating training data comprising the comparator variables and the label data, and training the machine learning model using the training data. The machine learning model is trained to approximate the label data, when the training data is applied as input data to the machine learning model.

The training of the machine learning model may be implemented in any suitable manner, such as by employing an optimization algorithm which uses the training data to minimize a suitable loss function, which loss function may be a function of the difference between an estimated and a true value of the response variable, in other words the label data, as previously described.

Regarding the label data, the assessment of the respiratory disease comprises, in some embodiments, a measure of the respective training subject's usage of the inhaler determined after the subsequent period, in other words in a period which follows the subsequent period.

In a non-limiting example, the measure of the respective training subject's usage of the inhaler determined after the subsequent period may, for instance, comprise an assessment of whether the respective training subject fulfils the above-described excessive rescue inhaler usage measure indicative of excessive rescue inhaler usage after the subsequent period.

Thus, the trained machine learning model can be used to predict future rescue inhaler usage based on comparing baseline and current/subsequent rescue inhaler usage, e.g. baseline and current/subsequent rescue inhaler usage in non-contiguous baseline and current periods respectively.

Excessive rescue inhaler usage, for instance, may be indicative of worsening of the subject's respiratory disease. The capability to predict future rescue inhaler usage may correspondingly provide a warning of, for example, an impending exacerbation. A clinical assessment may, in at least some embodiments, not be required to train the machine learning model because the rescue inhaler usage-based label data can comprise the measure of each of the training subjects' usage of the inhaler determined after the subsequent period.

Alternatively or additionally, the measure of the respective training subject's usage of the inhaler may comprise one or more parameters relating to airflow during an inhalation performed by the respective training subject using the inhaler. The one or more parameters relating to airflow may comprise, or consist of, at least one of a peak inhalation flow, an inhalation volume, a time to peak inhalation flow, and an inhalation duration. The determined inhaler usage may be used to predict the parameter, and thus predict the user's lung function, as previously described.

Alternatively or additionally, the assessment of the respiratory disease may comprise an approximation of a clinically determined indication of the status of the subject's respiratory disease, for example, a “moderate” or “severe” exacerbation as defined herein. The term “clinically determined indication” may refer to a clinical assessment of the subject made independently of inhaler usage data, for example by a health care provider.

Some embodiments of the method for training the machine learning model comprise, for each of a plurality of clinical assessment subjects, determining the baseline statistic; determining the subsequent statistic; generating the comparator variable, comprising comparing the subsequent statistic and the baseline statistic. The method further comprises obtaining further label data comprising a clinically determined indication of the status of the respective clinical assessment subject's respiratory disease. Further training data comprising the comparator variables and the further label data is then generated, and an adapted machine learning model is trained using the further training data.

In a non-limiting example, the label data included in the training data for training the machine learning model comprises the measure of each of the training subjects' usage of the inhaler determined after the subsequent period, but without any clinically determined indication.

Having trained the machine learning model using this label data, the thus trained model may be adapted, e.g. validated, using the above-described further training data comprising the further label data. Since the further label data comprises comprising the clinically determined indication of the status of the respective clinical assessment subject's respiratory disease, training the adapted model may provide an adapted machine learning model configured to predict an exacerbation in the subject's respiratory disease.

The adapted machine learning model may comprise input(s), including the comparator variable, which correspond to, e.g. are the same as, at least some of the input(s) used for training the (initial) machine learning model.

It is noted that there may be at least some overlap between the plurality of training subjects and the plurality of clinical assessment subjects, depending on whether the clinical assessment is made for any of the former. In cases of such overlap, determining the baseline statistic, determining the subsequent statistic, and generating the comparator variable need not be repeated.

In an alternative non-limiting example, a method for training the machine learning model comprises, for each of a plurality of clinical assessment subjects, providing one or more excessive rescue inhaler usage measures, e.g. one or more of the excessive rescue inhaler usage measures, such as a SABA burst, as defined in any of the above-described examples. The method further comprises obtaining further label data comprising a clinically determined indication of the status of the respective clinical assessment subject's respiratory disease. Further training data comprising the one or more excessive rescue inhaler usage measures and the further label data is then generated, and a further machine learning model is trained using the further training data. The further machine learning model is trained to approximate the further label data, when the further training data is applied as input data to the machine learning model.

In such an example, the clinically determined indication may comprise or consist of a clinically confirmed exacerbation of the subject's respiratory disease. Thus, the excessive rescue inhaler usage measure(s) may be applied to the further machine learning model in order to, for example, predict an exacerbation (due to the further machine learning model being trained to approximate such a clinically determined exacerbation). Such a prediction may be of the subject suffering an exacerbation of their respiratory disease in an exacerbation prediction period, as previously described.

In some embodiments a method for generating an assessment of a respiratory disease in a subject at a current point in time comprises: determining the above-described baseline statistic relating to usage of an inhaler in a baseline period, which inhaler is configured to deliver a rescue medicament to the subject, and has a use determination system configured to determine usage of the inhaler by the subject. The method also comprises determining the above-described current statistic relating to usage of the inhaler in a current period containing the current point in time, with an intervening period separating the current period from the baseline period. Generating a comparator variable comprises comparing the current statistic and the baseline statistic. The method further comprises generating the assessment based on the comparator variable.

In such embodiments, a model, e.g. a suitable linear or non-linear model, may be used to generate the assessment, but the model need not be a machine learning model. Such a model may, for example, be based on, or derived from, one or more of the machine learning models described above, rather than itself being constructed via machine learning techniques.

Further provided is a method for training a machine learning model for use in generating an assessment of a respiratory disease in a subject. This method comprises, for each of a plurality of training subjects, obtaining measurement data, comprising data relating to the training subject's usage of an inhaler. The inhaler is configured to deliver a rescue medicament to the training subject and has a use determination system configured to determine usage of the inhaler by the training subject, as previously described.

A baseline statistic relating to usage of the inhaler in a baseline period is determined. A subsequent statistic relating to usage of the inhaler in a subsequent period is also determined. An intervening period may separate the subsequent period from the baseline period, similarly to in the above-described embodiments of the method for generating the assessment of the subject's respiratory disease in which the baseline period is separated from the current period by the intervening period.

A comparator variable is generated for each of the plurality of training subjects. Generating the comparator variable comprises comparing the subsequent statistic and the baseline statistic.

The method further comprises labelling the measurement data according to whether the comparator variable exceeds a predetermined threshold, and training the machine learning model using the labelled measurement data for the plurality of training subjects. The machine learning model is thereby trained to generate an assessment of the respiratory disease.

The present disclosure also provides a computer program comprising computer program code which is configured, when the program is run on one or more physical computing devices, to cause the one or more physical computing devices to implement one or more of the above-described methods.

Similarly, the present disclosure provides one or more non-transitory computer readable media having a computer program stored thereon, the computer program comprising computer program code which is configured, when the computer program is run on one or more physical computing devices, to cause the one or more physical computing devices to implement one or more of the above-described methods.

A system for generating an assessment of a respiratory disease in a subject at a current point in time is also provided. The system comprises an inhaler for delivering a rescue medicament to the subject, the inhaler having a use determination system configured to determine a rescue inhalation performed by the subject using the inhaler. The system further comprises one or more processors configured to determine a baseline statistic relating to usage of the inhaler in a baseline period, and determine a current statistic relating to usage of the inhaler in a current period containing the current point in time. The one or more processors is or are also configured to generate a comparator variable. Generating the comparator variable comprises comparing the current statistic and the baseline statistic, as previously described.

An intervening period may separate the current period from the baseline period, as described above in relation to at least some embodiments of the methods.

The one or more processors is or are configured to generate the assessment based on the comparator variable.

The one or more processors may be implemented in any suitable way, and may, for example, include a general purpose processor, a special purpose processor, a DSP, a microcontroller, an integrated circuit, and/or the like that may be configured using hardware and/or software to perform the functions described herein for the one or more processors. The one or more processors may be included partially or entirely in the inhaler, a user device, and/or a server.

The one or more processors may be, for example, provided in the system along with, for instance, further electronic components, such as a power supply, e.g. a battery, and a memory.

In a non-limiting example, the one or more processors is or are at least partly included in a first processing module included in a user device, such as a a personal computer, a tablet computer, and/or a smart phone. In other non-limiting examples, the one or more processors is or are not included in a user device. The one or more processors (or at least part of thereof) may, for example, be provided in a server, e.g. a remote server. For example, the one or more processors may be implemented on any combination of the inhaler, the user device, and/or the remote server. As such, any combination of the functions or processing described with reference to the one or more processors may be performed by processor(s) residing on the inhaler, the user device, and/or a server.

In at least some embodiments, the one or more processors is or are configured to apply the comparator variable as an input to a trained machine learning model. Examples of such a trained machine learning model, and the training of such a machine learning model, have been described above in relation to the methods. The one or more processors is or are configured to generate, as an output of the trained machine learning model, the assessment of the respiratory disease in the subject.

In some embodiments, the system comprises a user interface, and the one or more processors is or are configured to control the user interface to communicate a notification based on the generated assessment.

The notification may, for example, comprise a warning and/or recommendation, e.g. a recommendation for the subject to seek medical attention and/or take some other pre-emptive step. Alternatively or additionally, the notification may be in the form of a prompt for prompting the subject to provide an indication of the status of their respiratory disease, as previously described.

The user interface may, for example, be configured to enable user-inputting of the indication, as well as to communicate the notification.

The user interface may, for example, comprise a first user interface configured to enable using-inputting of the indication, and a second user interface configured to, when controlled by the one or more processors, output the notification, e.g. the warning and/or the prompt.

The first and second user interface may, for instance, be included in the same user device.

In a non-limiting example, the user interface comprises a touchscreen. In such an example, the second user interface comprises the display of the touchscreen, and the first user interface comprises the touch inputting system of the touchscreen. Such a touchscreen enables facile user-inputting and prompting, and is thus particularly beneficial in the scenario in which the subject's respiratory disease is worsening, as indicated by the generated assessment.

As an alternative or in addition to the prompt being issued via the touchscreen, the second user interface may comprise a loudspeaker for issuing, when controlled by the one or more processors, an audible notification, e.g. prompt and/or warning.

In an embodiment, the user interface, e.g. the first user interface, is configured to provide a plurality of user-selectable respiratory disease status options. In this case, the indication is defined by user-selection of at least one of the status options.

The user interface may, for example, prompt the user or subject to provide the indication via a pop-up notification link to complete a short questionnaire.

In a non-limiting example, the user interface displays a questionnaire comprising questions whose answers correspond to the indication. The user, e.g. the subject or his/her health care provider, may input the answers to the questions using the user interface.

In some embodiments, the system comprises a memory, for example a memory for storing each indication inputted via the user interface. The indication may be subsequently retrieved, for example to support a dialogue between the subject and his/her healthcare provider. In this manner, the subject's recollection of a previous status of their respiratory disease need not be relied upon for the purposes of the dialogue.

The questionnaire may be relatively short, i.e. with relatively few questions, in order to minimize burden on the subject. The number and nature of the questions may nevertheless be such as to ensure that the indication enables the clinical condition of the subject, e.g. including the likelihood of the subject experiencing an exacerbation, to be reliably assessed.

Particular mention is made of inputting the indication in the form of a six-point/six-question questionnaire because the requirement for sufficient clinical information is balanced with avoiding placing too much burden on the subject, particularly as he/she may be suffering from worsening symptoms, as indicated by the generated assessment.

More generally, the object of the questionnaire is to ascertain a contemporaneous or relatively recent (e.g. within the past 24 hours) indication in order to obtain “in the moment” understanding of the subject's well-being (in respect of their respiratory disease) with a few timely questions which are relatively quickly answered. The questionnaire may be translated into the local language of the subject.

Conventional control questionnaires, and especially the most established being ACQ/T (Asthma Control Questionnaire/Test) in asthma, or CAT (COPD Assessment Test) in COPD tend to focus on patient recall of symptoms in the past. Recall bias, and a focus on the past instead of the present is likely to negatively influence their value for the purposes of predictive analysis.

The following is provided by way of non-limiting example of such a questionnaire. The subject may select from the following status options for each question: All of the time (5); Most of the time (4); Some of the time (3); A little (2); None (1).
1. How ‘often are you experiencing’, or ‘Rate your’ shortness of breath?
2. How ‘often are you experiencing’, or ‘Rate your’ coughing?
3. How ‘often are you experiencing’, or ‘Rate your’ wheezing?
4. How ‘often are you experiencing’, or ‘Rate your’ chest tightness?
5. How ‘often are you experiencing’, or ‘Rate your’ night symptoms/affecting sleep?
6. How ‘often are you experiencing’, or ‘Rate your’ limitation at work, school or home?

An alternative example questionnaire is also provided:
1. Are you having more respiratory symptoms than usual (Y/N)? If yes:
2 More chest tightness or shortness of breath (Y/N)?
3. More cough (Y/N)?
4. More wheezing (Y/N)?
5. Is it affecting your sleep (Y/N)?
6. Is it limiting your activities at home/work/school (Y/N)?

Still another example questionnaire is also provided:
1. Are you having more:
   chest tightness or shortness of breath? (Y/N)
   cough? (Y/N)
   wheezing? (Y/N)
2. Are you sleeping well? (Y/N)
3. Are you limiting your daily activities in any way? (Y/N)
4. Have you had an infection or allergen (e.g. cat, pollen) exposure? (Y/N)

Yet another example questionnaire is also provided:
1. Are you having:
   More chest tightness or shortness of breath? (Y/N)
   More cough? (Y/N)
   More wheezing? (Y/N)
2 Are you sleeping well? (Y/N)
3. Are you limiting your activities at home/work/school? (Y/N)
4. Have you had an infection? (Y/N)
   If yes, did you take any antibiotics and/or steroids? (Y/N)
5. Have you had an allergen (e.g. cat, pollen) exposure recently? (Y/N)
6 (Optional) What is your most recent hospital anxiety and depression scale (HADS) score?

The answers to the questions may, for example, be used to calculate a score, which score is included in, or corresponds to, the indication of the status of the respiratory disease being experienced by the subject.

In some embodiments, the user interface is configured to provide the status options in the form of selectable icons, e.g. emoji-type icons, checkboxes, a slider, and/or a dial. In this way, the user interface may provide a straightforward and intuitive way of inputting the indication of the status of the respiratory disease being experienced by the subject. Such intuitive inputting may be particularly advantageous when the subject himself/herself is inputting the indication, since the relatively facile user-input may be minimally hampered by any worsening of the subject's respiratory disease.

Any suitable user interface may be employed for the purpose of enabling user-input of the indication of the status of the respiratory disease being experienced, e.g. subjectively, by the subject. For example, the user interface may comprise or consist of a user interface of a user device. The user device may be, for example, a personal computer, a tablet computer, and/or a smart phone. When the user device is a smart phone, the user interface may, for instance, correspond to the touchscreen of the smart phone, as previously described.

In some non-limiting examples, the system may be further configured such that the indication can be inputted via the user interface when the user opts to so input the indication. Thus, the user, e.g. the subject, need not wait for the prompt in order to input the indication.

Alternatively or additionally, the one or more processors may be configured to issue the prompt based on no flags indicating worsening of the subject's condition are triggered during a predetermined time period, e.g. 7 days.

This may assist to a) ensure that there are no symptoms that the patient is having that the use determination system (use and/or inhalation parameter) is missing; and/or b) to capture if a patient is well (e.g. all ‘no’ answers to the above-described questionnaire) and that the indication and the rescue inhaler use and inhalation parameter data are thus aligned with each other; and/or c) as a way to capture whether and when the patient is recovering.

More generally, any embodiments described herein in respect of the methods, computer program, and non-transitory computer readable media, are applicable to the systems described herein, and any embodiments described in respect of the systems may be applied to the methods, computer program, and non-transitory computer readable media.

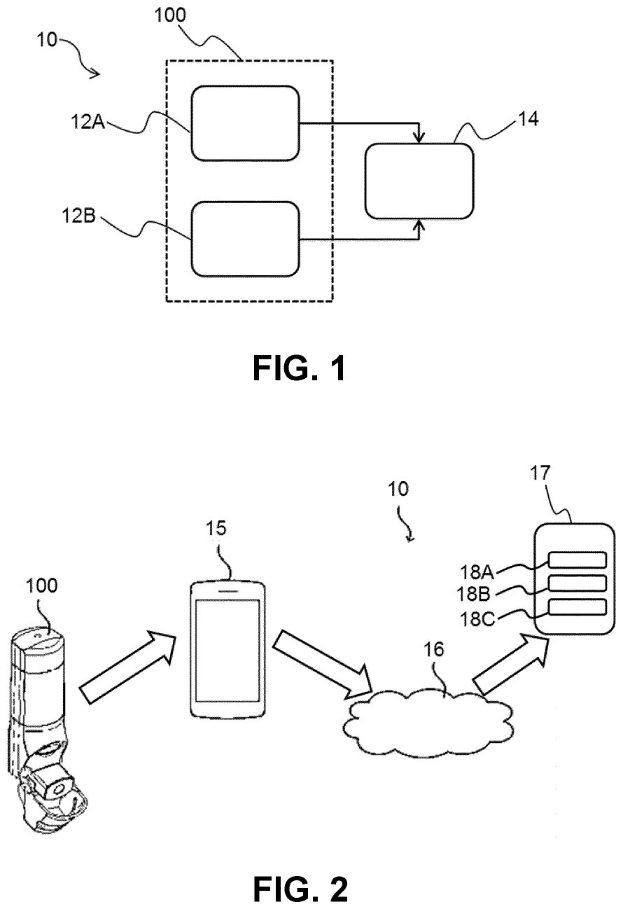

FIG. 1 shows a block diagram of a system 10 according to an embodiment. The system 10 comprises an inhaler 100 and one or more processors 14. The inhaler 100 may be used to deliver a rescue medicament, such as a SABA, to the subject. The SABA may include, for example, albuterol. The inhaler 100 may include a use determination system 12B, and optionally a sensor system 12A.

The system 10 may, for example, be alternatively termed “an inhaler assembly”.

The sensor system 12A may be configured to measure a value of the inhalation parameter from an inhalation performed by a subject using the inhaler 100. The sensor system 12A may, for example, comprise one or more sensors, such as one or more pressure sensors, temperature sensors, humidity sensors, orientation sensors, acoustic sensors, and/or optical sensors. The pressure sensor(s) may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

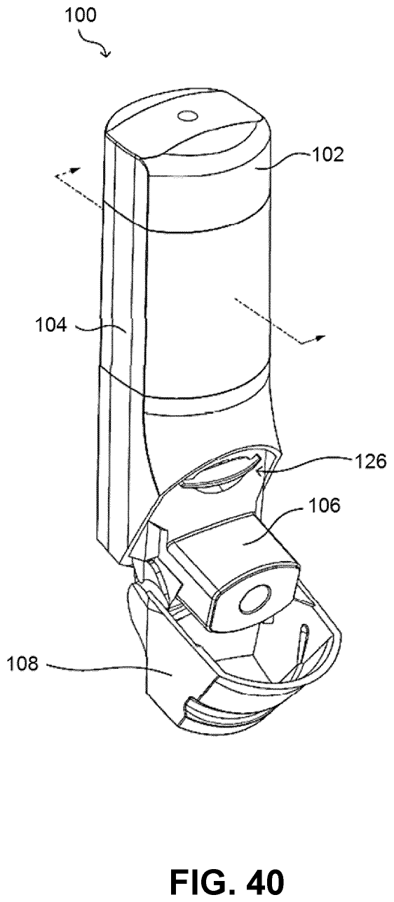

A pressure sensor(s) may be particularly suitable for measuring the parameter, since the airflow during inhalation by the subject may be monitored by measuring the associated pressure changes. As will be explained in greater detail with reference to FIGS. 40 to 44, a pressure sensor may be, for instance, located within or placed in fluid communication with a flow pathway through which air and the medicament is drawn by the subject during inhalation. Alternative ways of measuring the parameter, such as via a suitable flow sensor, will also be apparent to the skilled person.

Alternatively or additionally, the sensor system 12A may comprise a differential pressure sensor. The differential pressure sensor may, for instance, comprise a dual port type sensor for measuring a pressure difference across a section of the air passage through which the subject inhales. A single port gauge type sensor may alternatively be used. The latter operates by measuring the difference in pressure in the air passage during inhalation and when there is no flow. The difference in the readings corresponds to the pressure drop associated with inhalation.

Whilst not shown in FIG. 1, the system 10 may further comprise a further inhaler for delivering a maintenance medicament to the subject. The further inhaler may include a sensor system 12A and optionally a use determination system 12B that are respectively distinct from the optional sensor system 12A and the use determination system 12B of the inhaler 100. The sensor system 12A of the further inhaler may be configured to measure the value of the inhalation parameter from an inhalation performed by a subject using the further inhaler. For example, the sensor system 12A of the further inhaler may include a further pressure sensor, such as a further microelectromechanical system pressure sensor or a further nanoelectromechanical system pressure sensor, in order to measure the inhalation parameter during inhalation of the maintenance medicament.

Each inhalation may be associated with a decrease in the pressure in the airflow channel relative to when no inhalation is taking place. The point at which the pressure is at its lowest may correspond to the peak inhalation flow. The sensor system 12A may detect this point in the inhalation. The peak inhalation flow may vary from inhalation to inhalation, and may depend on the clinical condition of the subject. A peak inhalation flow which is declining over time may point to worsening of the subject's respiratory disease.

The pressure change associated with each inhalation may alternatively or additionally be used to determine an inhalation volume. This may be achieved by, for example, using the pressure change during the inhalation measured by the sensor system 12A to first determine the flow rate over the time of the inhalation, from which the total inhaled volume may be derived. Decreasing inhalation volumes over time may point to worsening of the subject's respiratory disease.

The pressure change associated with each inhalation may alternatively or additionally be used to determine an inhalation duration. The time may be recorded, for example, from the first decrease in pressure measured by the sensor system 12A, coinciding with the start of the inhalation, to the pressure returning to a pressure corresponding to no inhalation taking place. Shorter inhalation durations with time may point to decreased lung function, and therefore worsening of the subject's respiratory disease.

In an embodiment, the parameter includes the time to peak inhalation flow, e.g. as an alternative or in addition to the peak inhalation flow, the inhalation volume and/or the inhalation duration. This time to peak inhalation flow parameter may be recorded, for example, from the first decrease in pressure measured by the sensor system 12A, coinciding with the start of the inhalation, to the pressure reaching a minimum value corresponding to peak flow. A patient whose condition is declining may tend to take more time to achieve peak inhalation flow.

In a non-limiting example, the inhaler and/or the further inhaler may be configured such that, for a normal inhalation, the respective medicament is dispensed during approximately 0.5 s following the start of the inhalation. A subject's inhalation only reaching peak inhalation flow after the 0.5 s has elapsed, such as after approximately 1.5 s, may be partially indicative of the subject's lung condition being impaired.

In the non-limiting example shown in FIG. 1, the use determination system 12B is configured to register inhalation(s) performed by the subject. For the inhaler 100 configured to deliver the rescue medicament to the subject, the use determination system is configured to determine each rescue inhalation performed by the subject using the inhaler 100.

In a non-limiting example, the inhaler 100 may comprise a medicament reservoir (not shown in FIG. 1), and a dose metering assembly (not shown in FIG. 1) configured to meter a dose of the rescue medicament from the reservoir. The use determination system 12B may be configured to register the metering of the dose by the dose metering assembly, each metering being thereby indicative of the rescue inhalation performed by the subject using the inhaler 100. Accordingly, the inhaler 100 may be configured to monitor the number of rescue inhalations of the medicament, since the dose must be metered via the dose metering assembly before being inhaled by the subject. One non-limiting example of the metering arrangement will be explained in greater detail with reference to FIGS. 40 to 43.

Alternatively or additionally, the use determination system 12B may register each inhalation in different manners and/or based on additional or alternative feedback that are apparent to the skilled person. For example, the use determination system 12B may be configured to register an inhalation by the subject when the feedback from a sensor indicates that an inhalation by the user has occurred (e.g. when a pressure measurement or flow rate exceeds a predefined threshold associated with a successful inhalation). Further, in some examples, the use determination system 12B may be configured to register an inhalation when a switch of the inhaler or a user input of an external device (e.g. touchscreen of a smartphone) is manually actuated by the subject prior to, during or after inhalation.

A sensor (e.g. a pressure sensor) may, for example, be included in the use determination system 12B in order to register each inhalation. In such an example, the use determination system 12B and the sensor system 12A may employ respective sensors (e.g. pressure sensors), or a common sensor (e.g. a common pressure sensor) which is configured to fulfil both use-detecting and inhalation parameter sensing functions.

When a sensor is included in the use determination system 12B, the sensor may, for instance, be used to confirm that, or assess the degree to which, a dose metered via the dose metering assembly is inhaled by the user, as will be described in greater detail with reference to FIGS. 40 to 43.

In an embodiment, the sensor system 12A and/or the use determination system 12B includes an acoustic sensor. The acoustic sensor in this embodiment is configured to sense a noise generated when the subject inhales through the inhaler 100. The acoustic sensor may include, for example, a microphone.

In a non-limiting example, the inhaler 100 may comprise a capsule which is arranged to spin when the subject inhales though the device; the spinning of the capsule generating the noise for detection by the acoustic sensor. The spinning of the capsule may thus provide a suitably interpretable noise, e.g. rattle, for deriving use and/or inhalation parameter data.

An algorithm may, for example, be used to interpret the acoustic data in order to determine use data (when the acoustic sensor is included in the use determination system 12B) and/or the inhalation parameter relating to airflow during the inhalation (when the acoustic sensor is included in the sensor system 12A).

For instance, an algorithm as described by P. Colthorpe et al., “Adding Electronics to the Breezhaler®: Satisfying the Needs of Patients and Regulators”, Respiratory Drug Delivery 2018, 1, 71-80 may be used. Once the generated sound is detected, the algorithm may process the raw acoustic data to generate the use and/or inhalation parameter data.

The one or more processors 14 included in the system 10 can be configured in various ways. As schematically shown in FIG. 1 by the arrows between the sensor system 12A and the processor 14, the processor 14 may receive the inhalation parameter data from the optional sensor system 12A. In a similar way, the one or more processors 14 can receive usage data from the use determination system 12B.

In an embodiment, the one or more processors 14 is or are configured to determine a baseline statistic relating to usage of the inhaler 100 in a baseline period, and determine a current statistic relating to usage of the inhaler 100 in a current period containing the current point in time. The one or more processors 14 is or are also configured to generate a comparator variable. Generating the comparator variable comprises comparing the current statistic and the baseline statistic. The one or more processors 14 is or are configured to generate an assessment based on the comparator variable, as previously described.

An intervening period may separate the current period from the baseline period, as described above in relation to at least some embodiments of the methods.

In at least some embodiments, the one or more processors 14 is or are configured to apply the comparator variable as an input to a trained machine learning model. Examples of such a trained machine learning model, and the training of such a machine learning model, have been described above in relation to the methods. The one or more processors is or are configured to generate, as an output of the trained machine learning model, the assessment of the respiratory disease in the subject.

Whilst not visible in FIG. 1, the system 10 may include a user interface, and the one or more processors 14 is or are configured to control the user interface to communicate a notification based on the generated assessment.

The notification may, for example, comprise a warning and/or recommendation, e.g. a recommendation for the subject to seek medical attention and/or take some other pre-emptive step. Alternatively or additionally, the notification may be in the form of a prompt for prompting the subject to provide an indication of the status of their respiratory disease, as previously described.

More generally, the one or more processors 14 of the system 10 may be provided and implemented in any suitable manner. In a non-limiting example, the one or more processors 14 may be provided separately from the respective inhaler(s), in which case the one or more processors 14 receive(s) the number of rescue inhalations transmitted thereto from the use determination system 12B and optionally inhalation parameter data transmitted thereto from the sensor system 12A. By processing the data in such an external processing unit, such as in the processing unit of an external device, or in a server, e.g. a remote server, the battery life of the inhaler may be advantageously preserved.

In an alternative non-limiting example, the one or more processors 14 may be an integral part of the inhaler 100, for example contained within a main housing or top cap (not shown in FIG. 1) of the inhaler 100. In such an example, connectivity to an external device need not be relied upon.

It may also be contemplated that some of the functions of the one or more processors 14 may be performed by an internal processing unit included in the inhaler 100 and other functions of the one or more processors 14 may be performed by the external processing unit.

More generally, the system 10 may include, for example, a communication module (not shown in FIG. 1) configured to communicate the generated assessment to the subject and/or a healthcare provider, such as a clinician. The subject and/or the clinician may then take appropriate steps based on the generated assessment. When, for instance, a smart phone processing unit is included in the processor, the communication functions of the smart phone, such as SMS, email, Bluetooth®, etc., may be employed to communicate the generated assessment to the healthcare provider.

FIG. 2 shows a non-limiting example of a system 10. The system 10 includes the inhaler 100, an external device 15 (e.g. a mobile device), a public and/or private network 16 (e.g. the internet, a cloud network, etc.), and a personal data storage device 17. The external device 15 may, for example, include a smart phone, a personal computer, a laptop, a wireless-capable media device, a media streaming device, a tablet device, a wearable device, a Wi-Fi or wireless-communication-capable television, or any other suitable internet protocol-enabled device. For example, the external device 15 may be configured to transmit and/or receive RF signals via a Wi-Fi communication link, a Wi-MAX communications link, a Bluetooth® or Bluetooth® Smart communications link, a near field communication (NFC) link, a cellular communications link, a television white space (TVWS) communication link, or any combination thereof. The external device 15 may transfer data through the public and/or private network 16 to the personal data storage device 17.

The inhaler 100 may include a communication circuit, such as a Bluetooth® radio, for transferring data to the external device 15.

The inhaler 100 may also, for example, receive data from the external device 15, such as, for example, program instructions, operating system changes, dosage information, alerts or notifications, acknowledgments, etc.

The external device 15 may include at least part of the one or more processors 14, and thereby process, analyze and/or communicate the usage of the inhaler 100 determined by the use determination system 12B, and optionally the inhalation parameter data from the sensor system 12A. For example, the external device 15 may process the usage data such as to determine the current and/or baseline statistic, generate the comparator variable, and generate the assessment measure, as represented by block 18A. Such information may be provided to the personal data storage device 17 for remote storage thereon.

In some non-limiting examples, the external device 15 may also process the data to identify no-inhalation events, low inhalation events, good inhalation events, excessive inhalation events and/or exhalation events, as represented by block 18B. The external device 15 may also process the data to identify underuse events, overuse events and optimal use events, as represented by block 18C. The external device 15 may, for instance, process the data to estimate the number of doses delivered and/or remaining and to identify error conditions, such as those associated with a timestamp error flag indicative of failure of the subject to inhale a dose of the medicament which has been metered by the dose metering assembly. The external device 15 may include a display and software for visually presenting the usage parameters through a graphical user interface.

Although illustrated as being stored on the personal data storage device 17, in some examples, at least some of the generated assessment, as represented by block 18A, the no inhalation events, low inhalations events, good inhalation events, excessive inhalation events and/or exhalation events, as represented by block 18B, and/or the underuse events, overuse events and optimal use events, as represented by block 18C, may be stored on the external device 15.

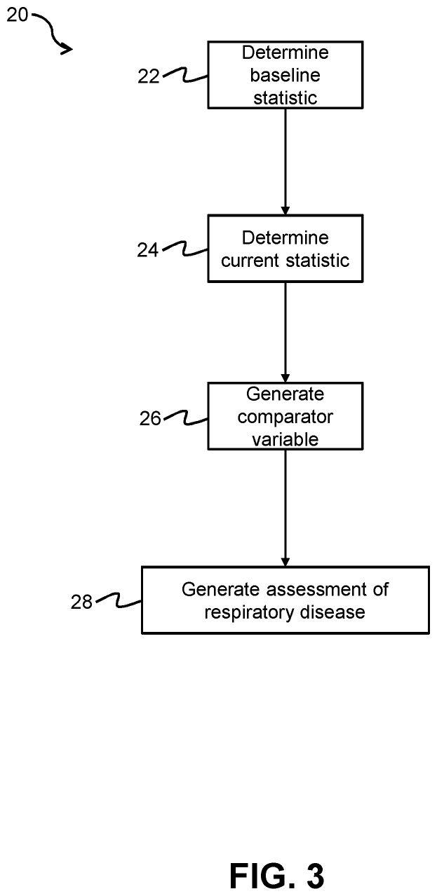

FIG. 3 provides a flowchart of a method 20 according to an example. The method 20 is for generating an assessment of a subject's respiratory disease at a current point in time. The method 20 comprises determining 22 a baseline statistic relating to usage of an inhaler in a baseline period. The inhaler is configured to deliver a rescue medicament to the subject, and has a use determination system configured to determine usage of the inhaler by the subject, as previously described.

The method 20 also comprises determining 24 a current statistic relating to usage of the inhaler in a current period containing the current point in time.

An intervening period may separate the current period from the baseline period, such that the baseline period and the current period are non-contiguous, as previously described. The intervening period may have a fixed duration. In some embodiments, the duration of the intervening period is 3 to 15 days, preferably about 7 days.

The method 20 further comprises generating 26 a comparator variable. Generating 26 the comparator variable in this example comprises comparing the current statistic and the baseline statistic. The assessment of the respiratory disease is generated in step 28. The assessment of the respiratory disease is based on the comparator variable.

It is noted that the order of operations depicted in FIG. 3 is not intended to be limiting, and the method 20 can be implemented in any suitable order, such as by the determining 24 the current statistic being carried out prior to the determining 22 the baseline statistic. The same applies to the other flowcharts provided in the present disclosure. Various optional steps are depicted in the flowcharts, as indicated in some cases by hashed boxes and/or arrows.

The generating 28 the assessment of the respiratory disease can be implemented in any suitable manner. In at least some embodiments, a model, e.g. a suitable linear or non-linear model, may be used to generate the assessment, but the model need not itself be a machine learning model. Such a model may, for example, be based on, or derived from, one or more of the machine learning models described above, rather than itself being constructed via machine learning techniques.

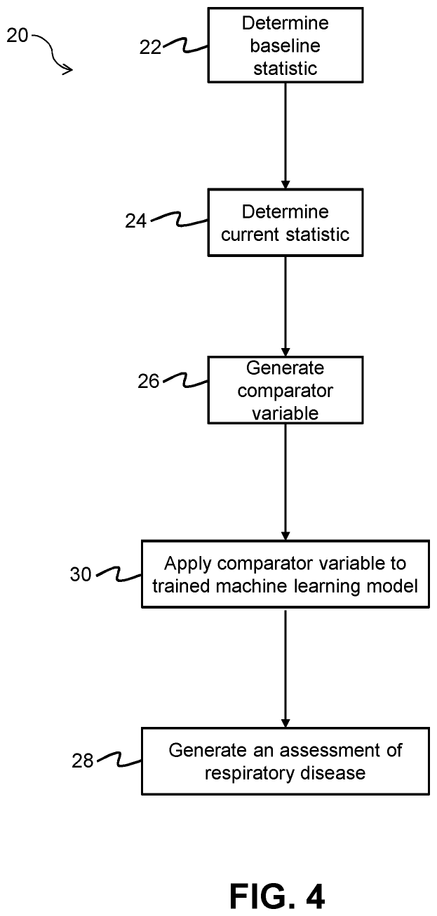

However, the method 20 shown in FIG. 4 further comprises applying 30 the comparator variable to a trained machine learning model. In this case, generating 28 the assessment of the respiratory disease involves the assessment being generated as an output of the trained machine learning model.

The baseline statistic may comprise, or consist of, one or more of a baseline average number of rescue inhalations using the inhaler per unit time, a baseline standard deviation of the number of rescue inhalations using the inhaler per unit time, and a baseline coefficient of variance of the number of rescue inhalations per unit time, calculated over the baseline period, as previously described.

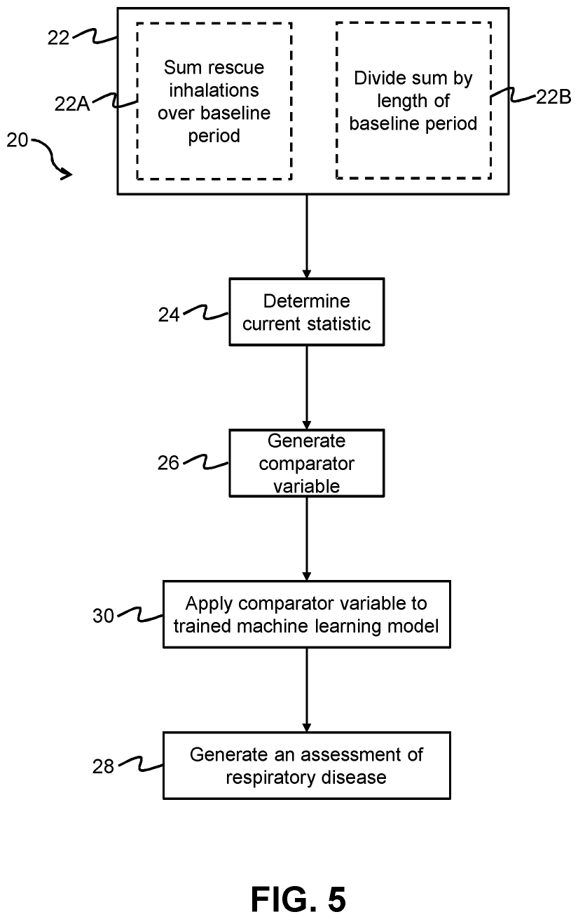

FIG. 5 depicts an exemplary method 20 in which determining 22 the baseline statistic comprises summing 22A the rescue inhalations over the baseline period, and dividing 22B the sum by the length of the baseline period. For example, the length of the baseline period may be a certain number of days, such as 10 to 30 days, preferably about 10 days or about 11 days or about 12 days or about 13 days or about 14 days or about 15 days or about 16 days or about 17 days or about 18 days or about 19 days or about 20 days or about 21 days or about 22 days or about 23 days or about 24 days or about 25 days, most preferably about 13 days or about 20 days. Thus, determining 22 the baseline statistic in this non-limiting example comprises determining 22A, 22B the mean number of daily rescue inhalations in the baseline period.

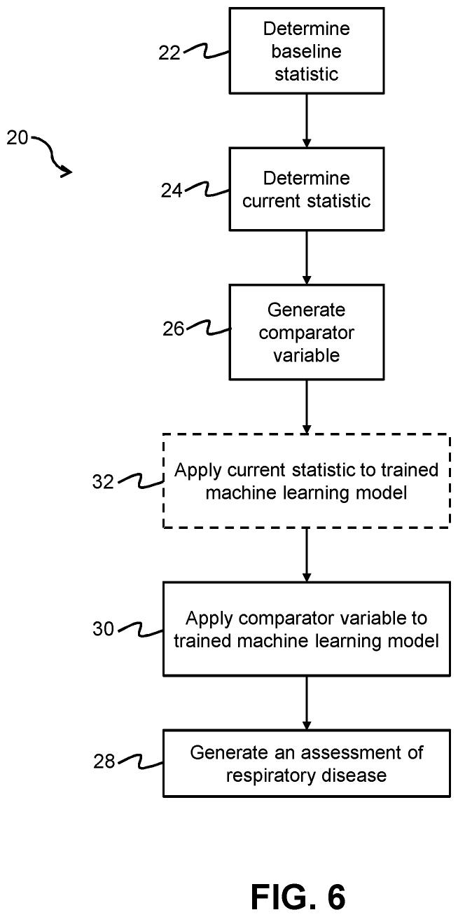

A plurality of inputs may be used in the method 20 in order to enable generating 28 of the assessment. The exemplary method 20 depicted in FIG. 6 comprises applying 32 the current statistic to the trained machine learning model, as well as applying 30 the comparator variable to the trained machine learning model. Thus, generating 28 the assessment is based on the current statistic and the comparator variable.

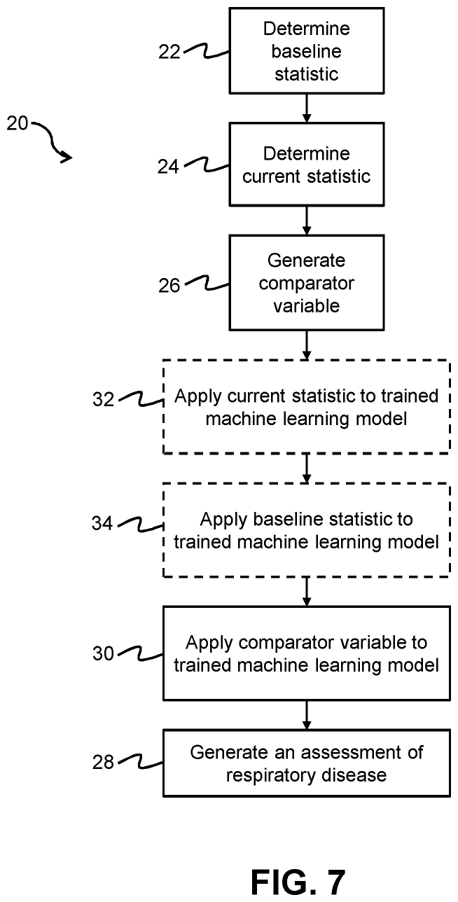

FIG. 7 depicts another example in which generating 28 the assessment is based on a plurality of inputs. In this non-limiting example, the method 20 comprises applying 32 the current statistic to the trained machine learning model, applying 34 the baseline statistic to the trained machine learning model, and applying 30 the comparator variable to the trained machine learning model. Thus, the trained machine learning model applies the current statistic and the baseline statistic themselves as inputs, in addition to the comparator variable being generated 26 by comparing the current statistic and the baseline statistic.

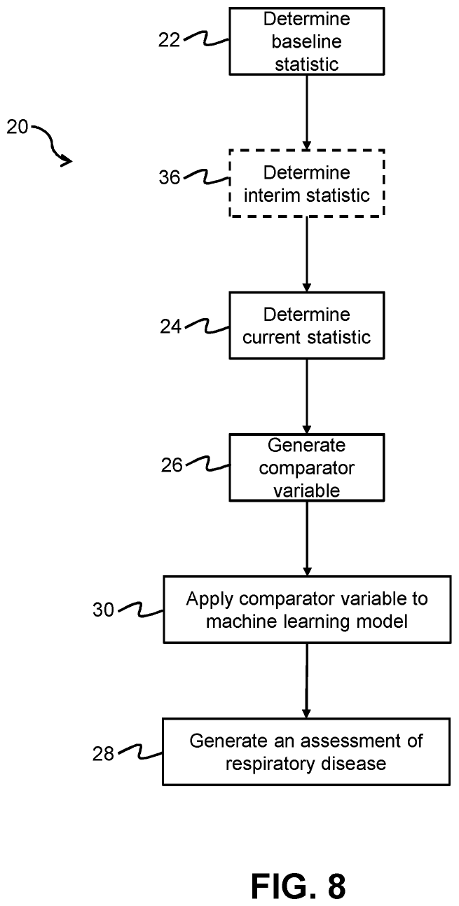

When the intervening period is provided to separate the baseline period and the current period, the method 20 may, as shown in FIG. 8, comprise determining 36 an interim statistic relating to usage of the inhaler in the intervening period.

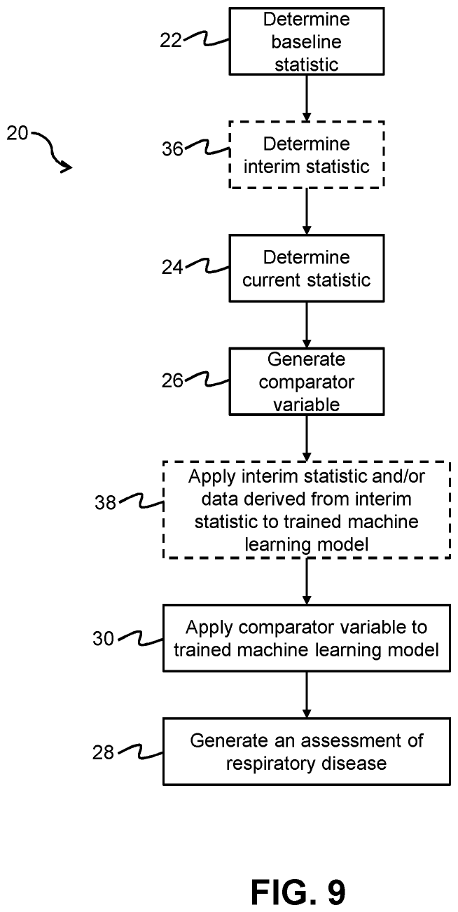

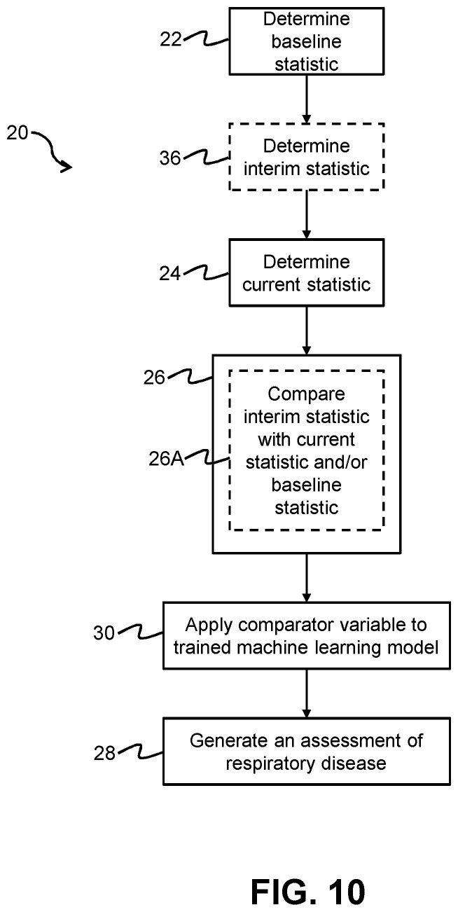

The interim statistic and/or data derived from the interim statistic can be, for example, applied 38 as an input to the machine learning model, as shown in FIG. 9. Alternatively or additionally, the generating 26 the comparator variable may further comprise comparing 26A the interim statistic with the current statistic and/or the baseline statistic, as shown in FIG. 10. Thus, the interim statistic can be used in the generating 28 of the assessment in various ways. By including the interim statistic in the method 20, the assessment may be additionally guided by a more recent trend in inhaler usage than provided via the baseline statistic.

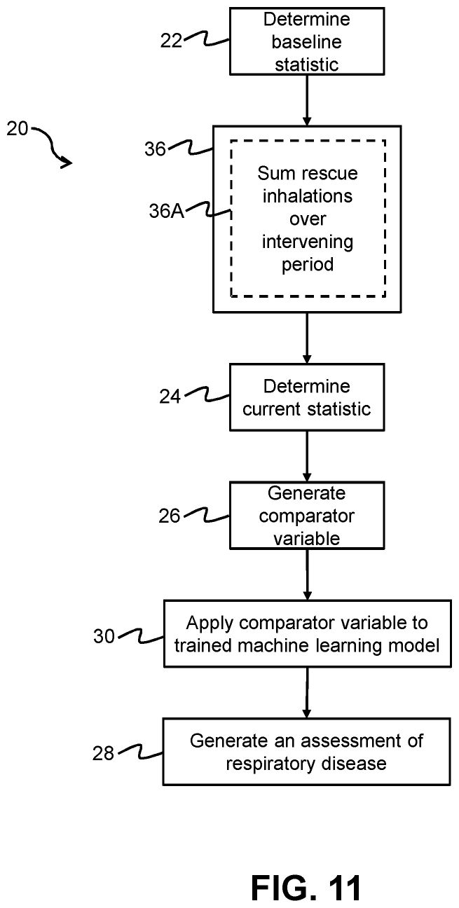

The interim statistic can be determined 36 in any suitable manner, provided that the interim statistic is indicative of the subject's usage of the inhaler during the intervening period. In the exemplary method 20 depicted in FIG. 11, determining 36 the interim statistic comprises determining 36A a total intervening number of rescue inhalations summed over the intervening period.

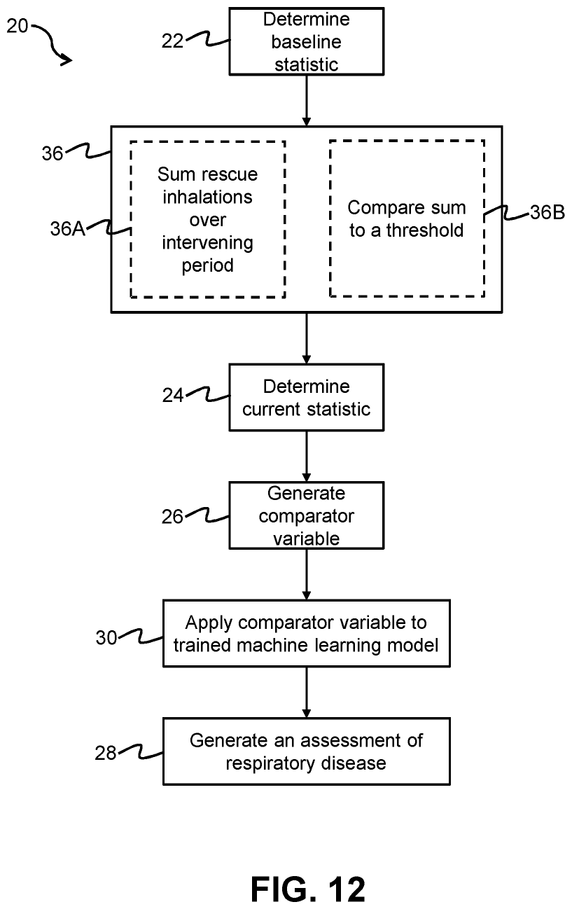

The total intervening number of rescue inhalations summed over the intervening period may itself be used in the generating 28 the assessment, for example in the various ways described above in relation to FIGS. 9 and 10. Alternatively, determining 36 the interim statistic may comprise determining 36A the total intervening number of rescue inhalations summed over the intervening period, and comparing 36B the sum to a given threshold, as shown in FIG. 12. In such an example, the interim statistic may comprise a value indicative of whether the sum reaches, exceeds, or is below such a given threshold.

Alternatively or additionally, the interim statistic may comprise, or consist of, one or more of an interim average number of rescue inhalations using the inhaler per unit time, an interim standard deviation of the number of rescue inhalations using the inhaler per unit time, and an interim coefficient of variance of the number of rescue inhalations per unit time, calculated over the intervening period, as previously described.

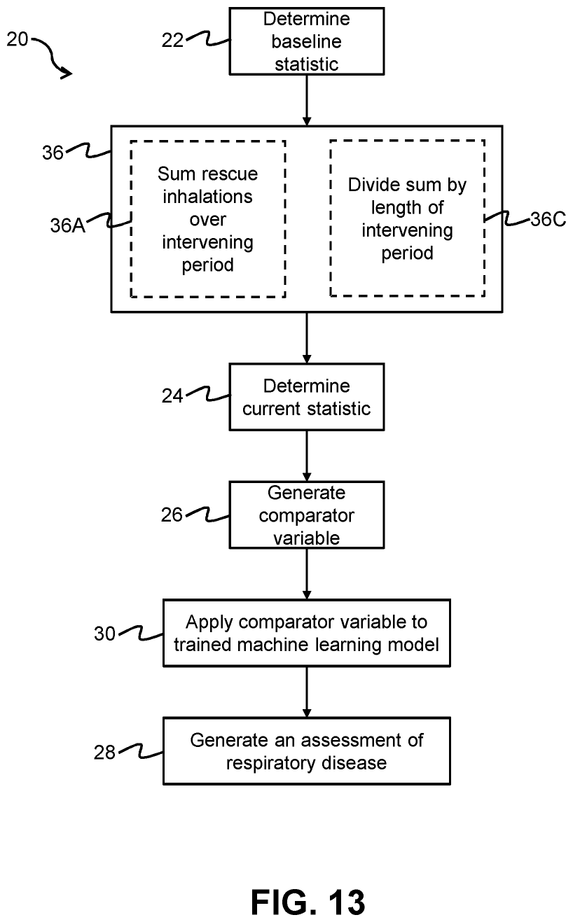

In the non-limiting example shown in FIG. 13, the determining 36 the interim statistic comprises determining 36A the total intervening number of rescue inhalations summed over the intervening period, and dividing 36C the sum by the length of the intervening period. For example, the length of the intervening period may be a certain number of days, such as 3 to 15 days, preferably about 7 days. Thus, determining 36 the interim statistic in this non-limiting example comprises determining 36A, 36C the mean number of daily rescue inhalations in the intervening period.

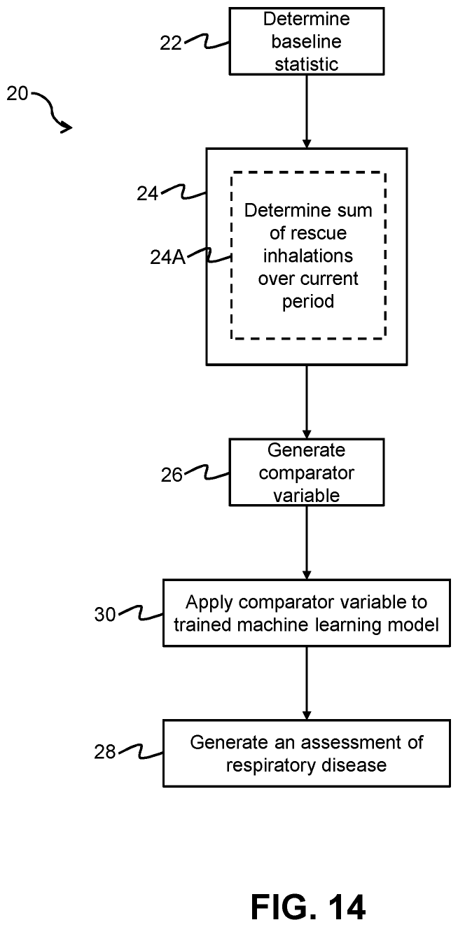

Determining 24 the current statistic may, for instance, comprise determining 24A the total current number of rescue inhalations summed over the current period, as depicted in FIG. 14. The total current number of rescue inhalations can, for example, itself be applied 32 as an input to the machine learning model, for instance as shown in FIG. 7.

The current statistic may comprise, or consist of, one or more of a current average number of rescue inhalations using the inhaler per unit time, a current standard deviation of the number of rescue inhalations using the inhaler per unit time, and a current coefficient of variance of the number of rescue inhalations per unit time, calculated over the current period, as previously described.

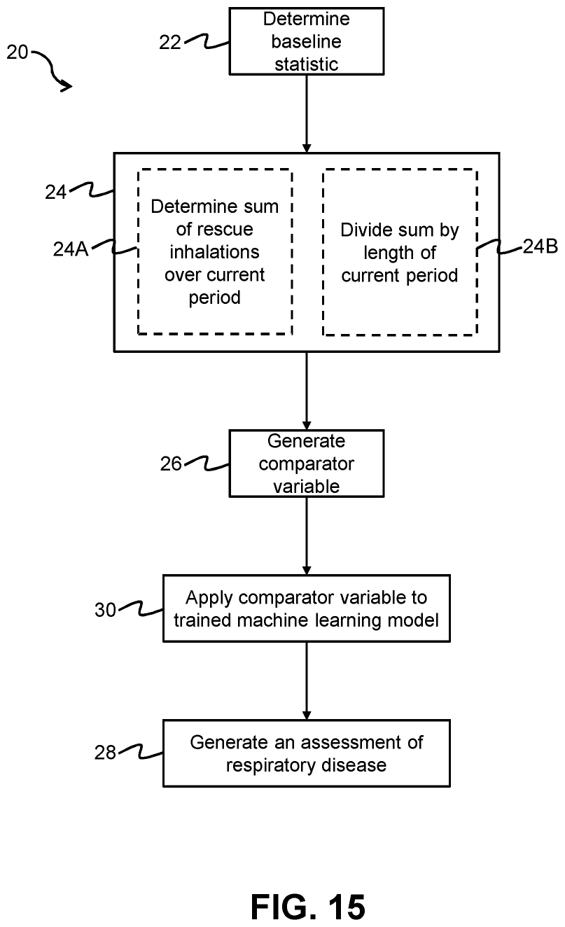

In the non-limiting example shown in FIG. 15, the determining 24 the current statistic comprises determining 24A the total current number of rescue inhalations summed over the current period, and dividing 24B the sum by the length of the current period. For example, the length of the current period may be a certain number of days, such as 1 to 5 days, preferably about 2 days. Thus, the determining 24 the current statistic in this non-limiting example comprises determining 24A, 24B the mean number of daily rescue inhalations in the current period.

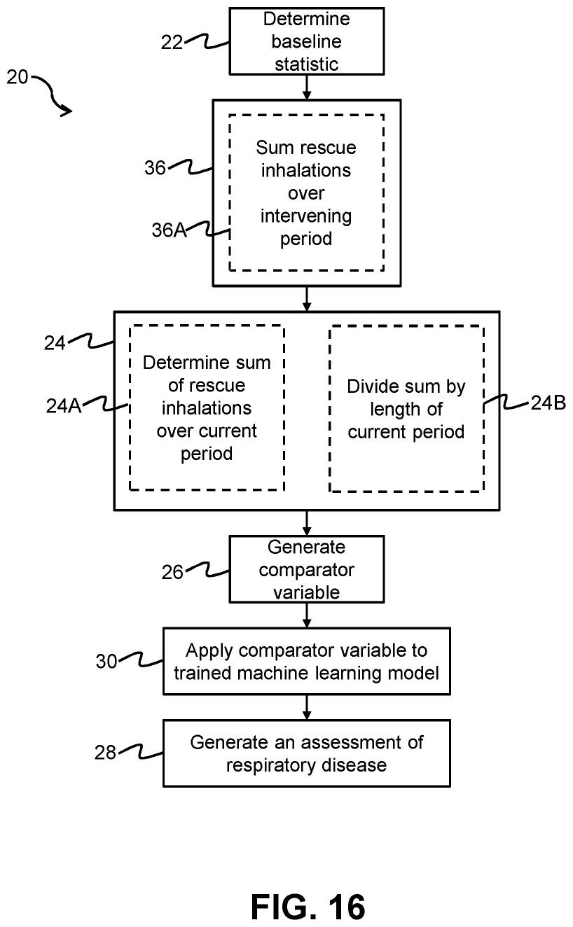

In the exemplary method 20 depicted in FIG. 16, the determining 24 the current statistic comprises determining 24A, 24B the mean number of daily rescue inhalations in the current period, and the determining 36 the interim statistic comprises determining 36A the total intervening number of rescue inhalations summed over the intervening period.

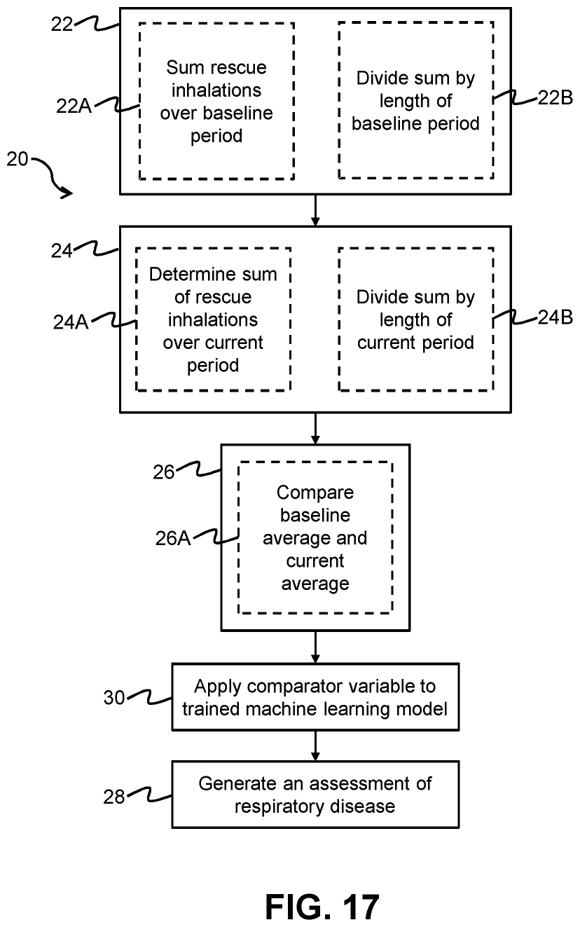

Turning to the non-limiting example depicted in FIG. 17, the determining 22 the baseline statistic comprises summing 22A the rescue inhalations over the baseline period, and dividing 22B the sum by the length of the baseline period; and the determining 24 the current statistic comprises determining 24A the total current number of rescue inhalations summed over the current period, and dividing 24B the sum by the length of the current period.

In this case, generating 26 the comparator variable can comprise comparing 26A the baseline average, in this example baseline mean, and the current average, in this example current mean.

The number of days of the baseline period and the current period may, for example, be used for the dividing steps 22B, 24B, such that the comparing 26A step involves comparing the baseline daily average number of inhalations and the current daily average number of inhalations.

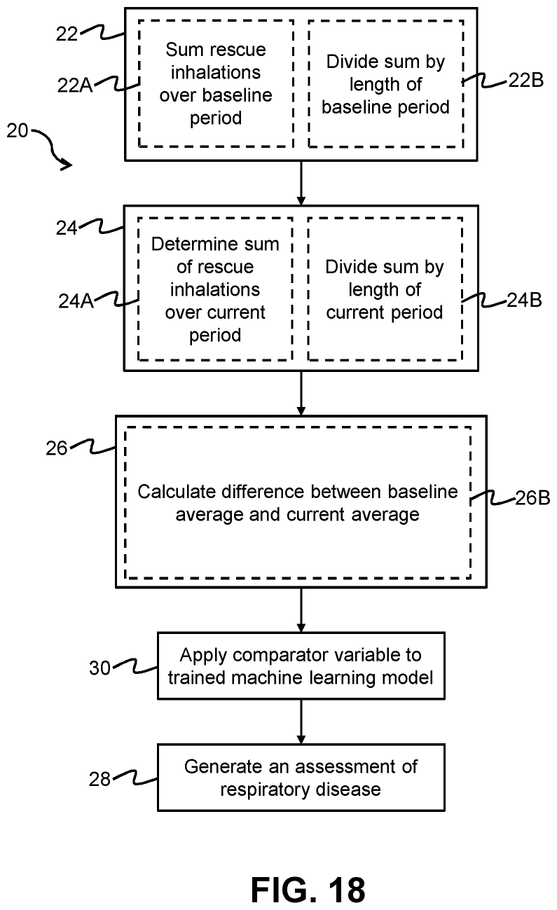

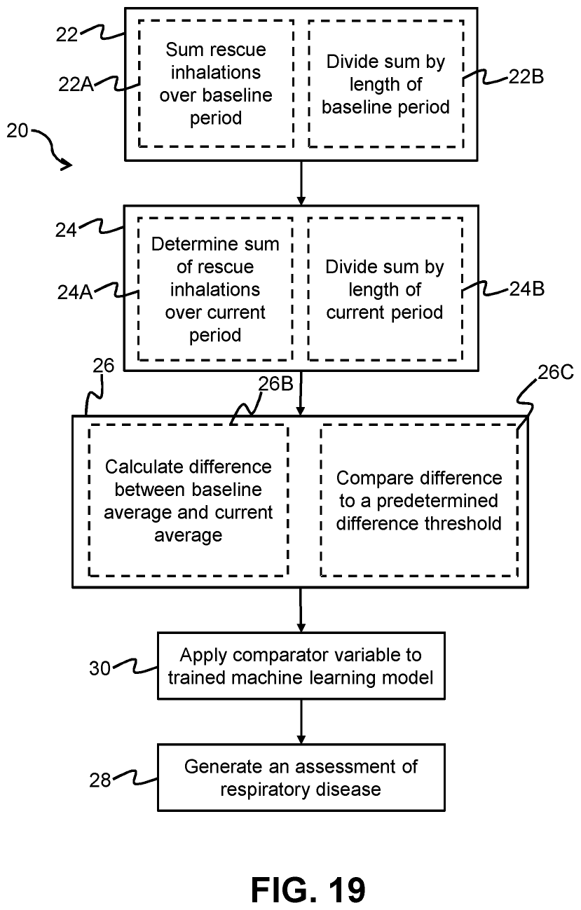

Turning to FIG. 18, the comparing 26A the baseline average and the current average comprises calculating 26B a difference between the mean daily number of inhalations in the baseline period and the mean daily number of inhalations in the current period. This difference may, for example, be compared 26C to a predetermined difference threshold, as shown in FIG. 19.

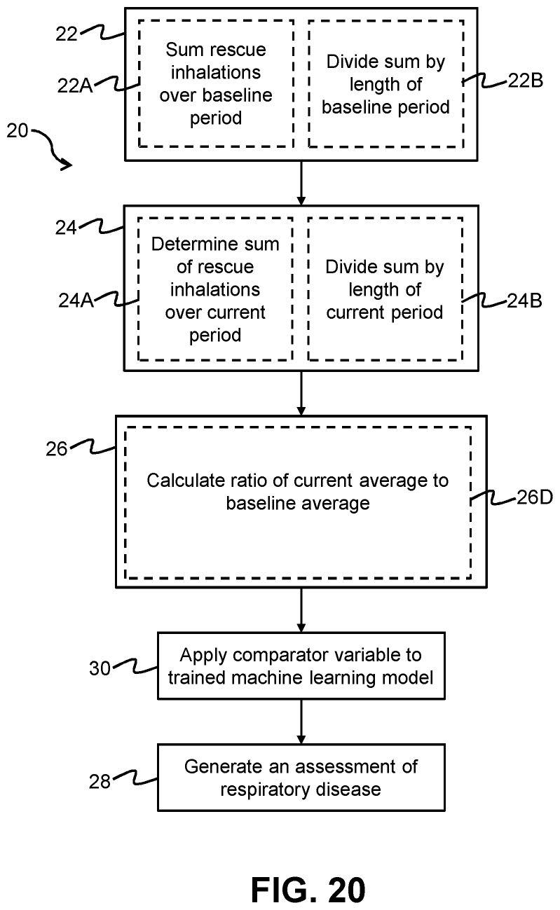

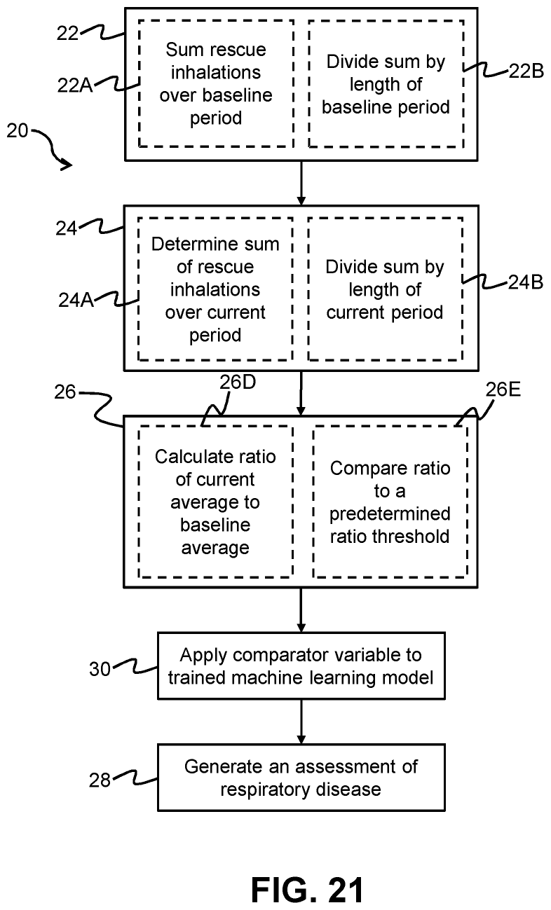

Alternatively or additionally, the comparing 26A the baseline average, in this example baseline mean, and the current average, in this example current mean comprises calculating 26D a ratio of the current average to the baseline average, as shown in FIG. 20. This ratio may, for example, be compared at step 26E to a predetermined ratio threshold, as shown in FIG. 21.

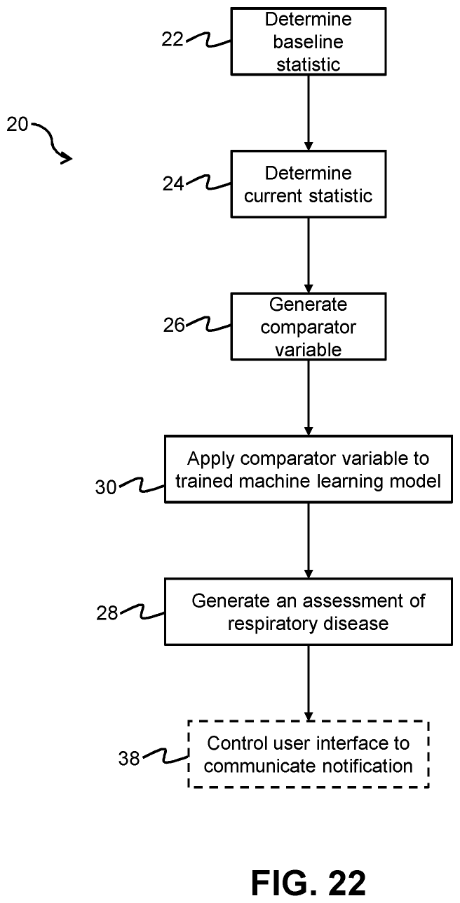

As shown in FIG. 22, the method 20 may comprise controlling 38 a user interface to communicate a notification based on the generated assessment of the subject's respiratory disease. The notification may, for example, comprise a warning and/or recommendation, e.g. a recommendation for the subject to seek medical attention and/or take some other pre-emptive step. Alternatively or additionally, the notification may be in the form of a prompt for prompting the subject to provide an indication of the status of their respiratory disease, as previously described.

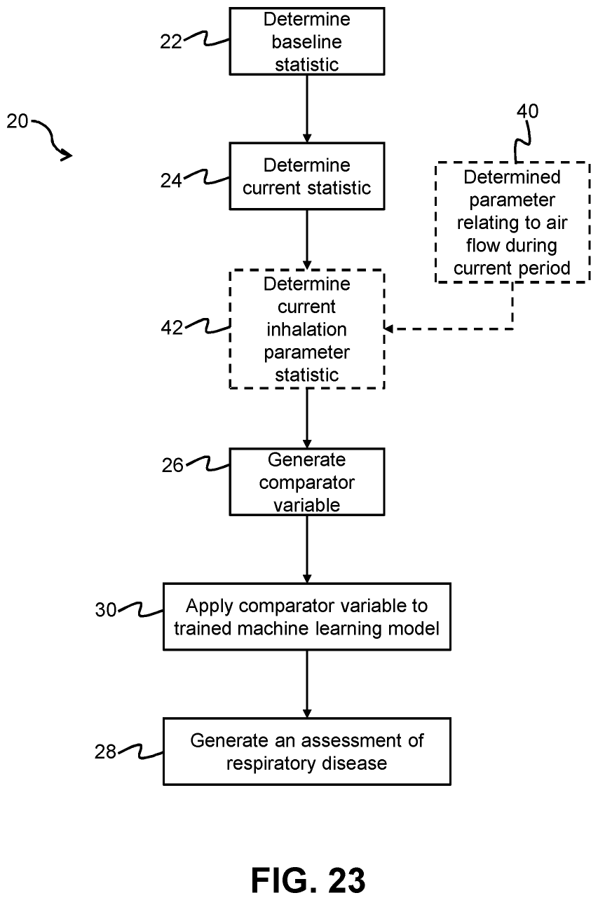

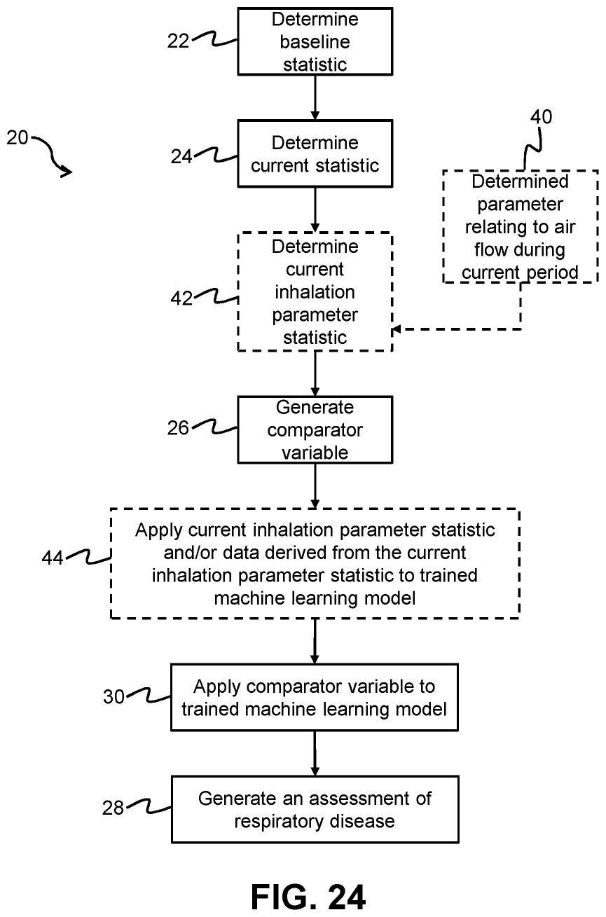

The exemplary method 20 depicted in FIG. 23 comprises determining 42 a current inhalation parameter statistic from a determined 40 parameter relating to airflow during an inhalation performed by the subject during the current period. The current inhalation parameter statistic and/or data derived from the current inhalation parameter statistic may, for example, be applied 44 as an input or inputs to the trained machine learning model, as shown in FIG. 24.

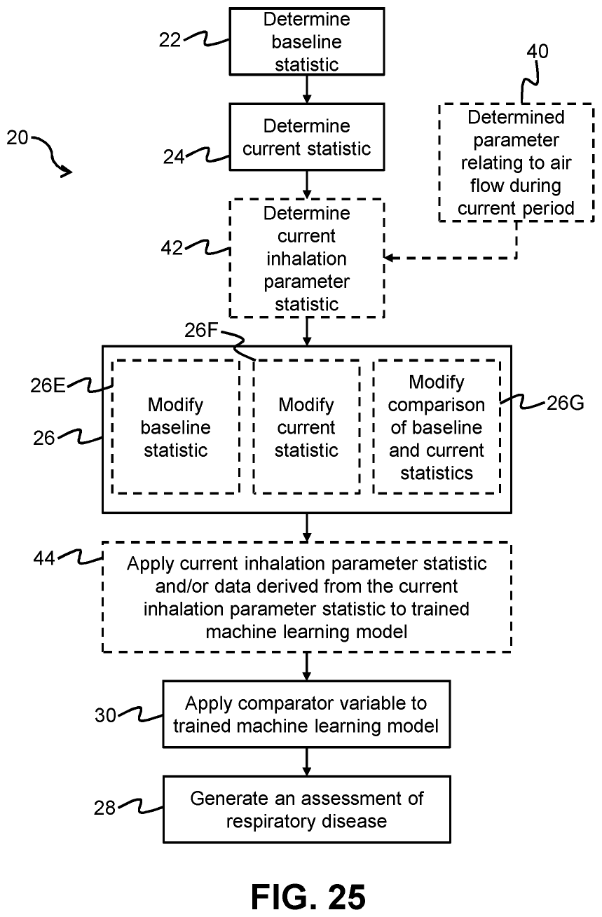

Alternatively or additionally, generating 26 the comparator variable may comprise modifying the baseline statistic in step 26E, the current statistic in step 26F and/or the comparison of the baseline and current statistics in step 26G with the current inhalation parameter statistic, as shown in FIG. 25.

Thus, the assessment may be generated 28 in light of the current inhalation parameter statistic, with the latter adding additional information concerning the current lung function of the subject.

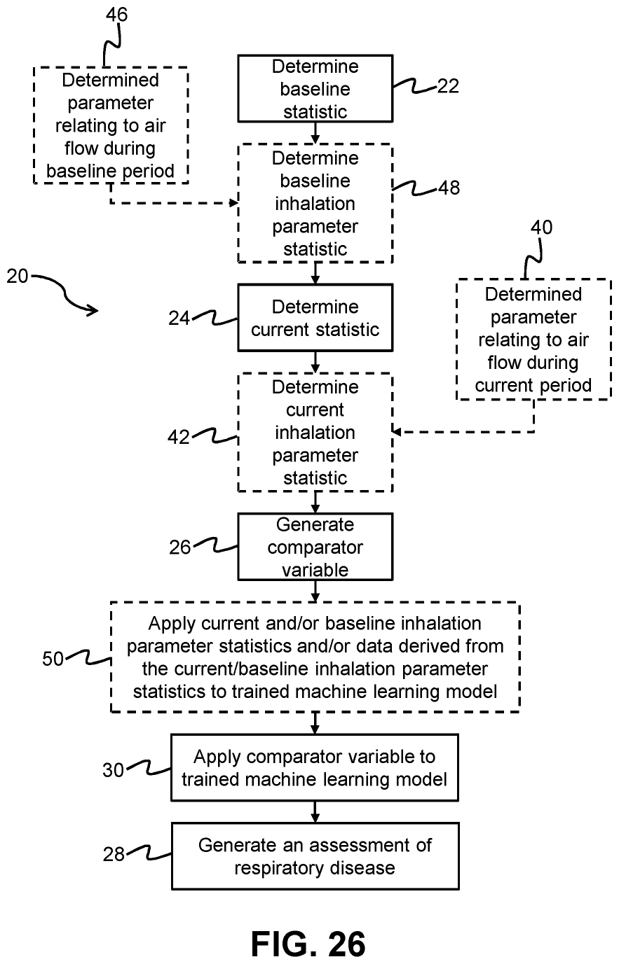

Turning to FIG. 26, the depicted exemplary method 20 comprises determining 48 a baseline inhalation parameter statistic from a determined 46 parameter relating to airflow during an inhalation performed by the subject using an inhaler during the baseline period. In this non-limiting example, the method 20 further comprises applying 50 the current and/or baseline inhalation parameter statistic(s) and/or data derived from the current/baseline inhalation parameter statistic as an input or input(s) to the machine learning model. Thus, the assessment may be partly based on the subject's baseline inhalation data indicative of their baseline lung function.

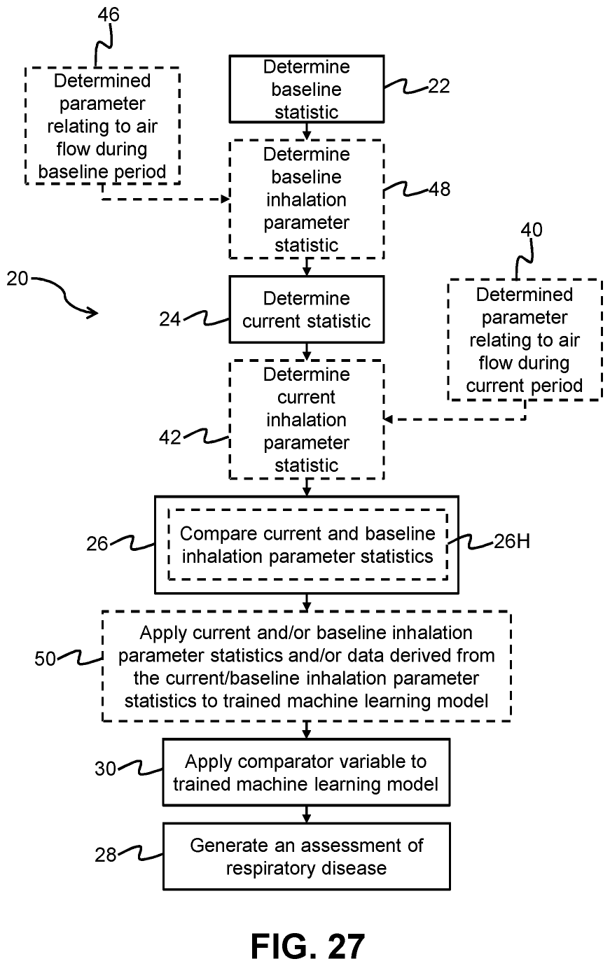

Alternatively or additionally, generating 26 the comparator variable further comprises comparing 26H the current inhalation parameter statistic and the baseline inhalation parameter statistic, as shown in FIG. 27. Thus, the generating 28 of the assessment may be partly based on a comparison between the subject's current inhalation data and their baseline inhalation data.

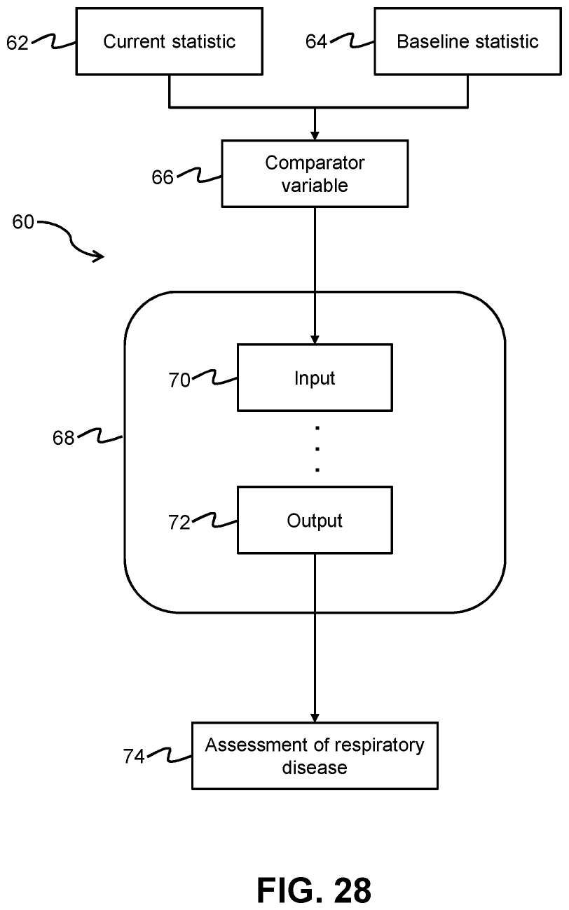

FIG. 28 provides a block diagram of machine learning model-comprising process 60. Blocks 62 and 64 correspond to the current statistic and the baseline statistic respectively. Block 66 represents generating the comparator variable comprising comparing the current statistic and the baseline statistic. The comparator variable is provided as an input to the trained machine learning model 68. The input to the model is represented in FIG. 28 by block 70. The machine learning model 68 provides an output at block 72. Block 74 represents the generated assessment of the subject's respiratory disease.

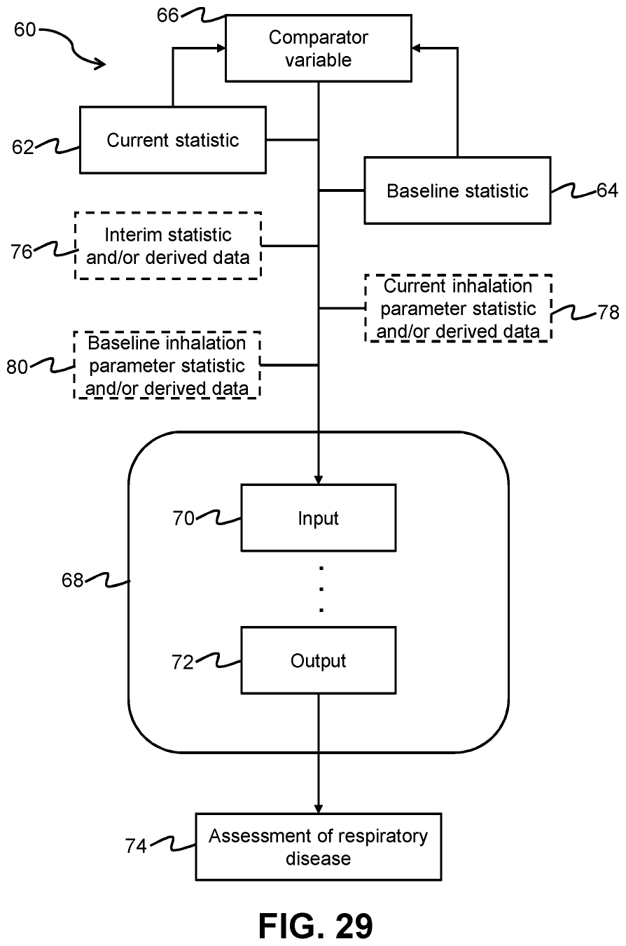

FIG. 29 provides a more elaborate block diagram of machine learning model-comprising process 60 in which the comparator variable 66 and various further statistics and/or data are provided as inputs 70 to the trained machine learning model 68. In particular, one or more of the above-described current statistic, baseline statistic, interim statistic and/or data derived from the interim statistic, current inhalation parameter statistic and/or data derived from the current inhalation parameter statistic, and baseline inhalation parameter statistic and/or data derived from the baseline inhalation parameter statistic can be provided as additional input(s) to the trained machine learning model 68, as previously described.

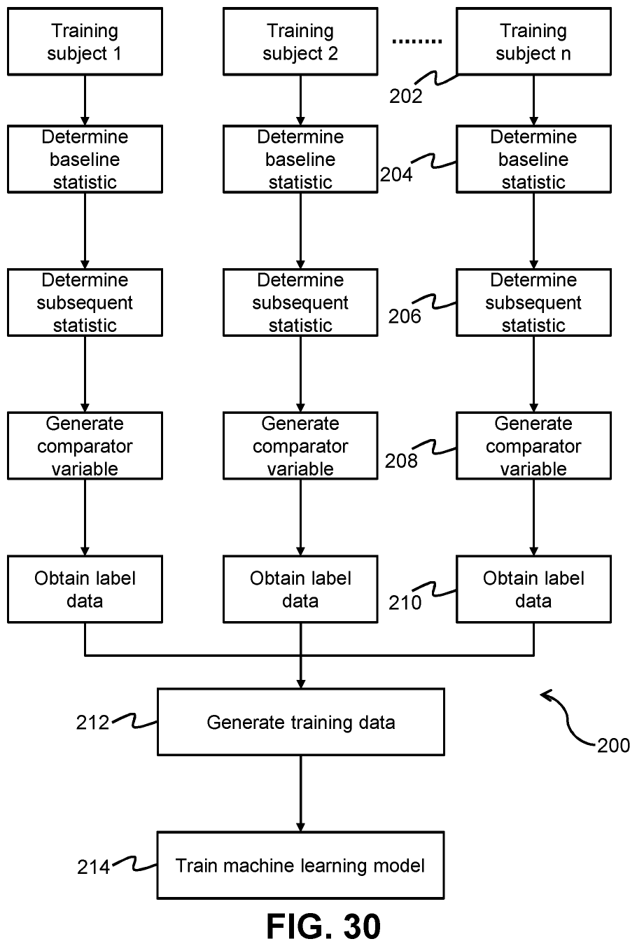

FIG. 30 shows a method 200 for training a machine learning model. The thus trained machine learning model may, for example, be utilized in any of the above-described methods 20 which employ such a trained machine learning model for generating the assessment of the subject's respiratory disease.

The method 200 for training the machine learning model comprises, for each of a plurality of training subjects 202, determining 204 a baseline statistic relating to usage of an inhaler in a baseline period. The inhaler is configured to deliver a rescue medicament to the training subject, and has a use determination system configured to determine usage of the inhaler by the training subject, as previously described.

The method 200 also comprises, for each of the plurality of training subjects 202, determining 206 a subsequent statistic relating to usage of the inhaler in a subsequent period.

An intervening period may separate the subsequent period from the baseline period, similarly to in the above-described embodiments of the method for generating the assessment of the subject's respiratory disease in which the baseline period is separated from the current period by the intervening period.

In the training method 200, a comparator variable is generated 208 for each of the plurality of training subjects. Generating 208 the comparator variable comprises comparing the subsequent statistic and the baseline statistic.

For each of the training subjects, label data, comprising an assessment of the respiratory disease in the respective training subject, is obtained at step 210. It is emphasized that the steps of the method 200 shown in FIG. 30 can be implemented in any suitable order, for example by the obtaining 210 of the label data being implemented prior to determining 204 the baseline statistic, and so on. Moreover, the order of operations need not be the same between different training subjects.

The method 200 further comprises generating 212 training data comprising the comparator variables and the label data, and training 214 the machine learning model using the training data.

The training 214 of the machine learning model may be implemented in any suitable manner, such as by employing an optimization algorithm which uses the training data to minimize a suitable loss function, which loss function may be a function of the difference between an estimated and a true value of the response variable, in other words the label data, as previously described.

Regarding the obtaining 210 of the label data, the assessment of the respiratory disease comprises, in some embodiments, a measure of the respective training subject's usage of the inhaler determined after the subsequent period, in other words in a period which follows the subsequent period.

In a non-limiting example, the measure of the respective training subject's usage of the inhaler determined after the subsequent period may, for instance, comprise an assessment of whether the respective training subject fulfils the above-described excessive rescue inhaler usage measure indicative of excessive rescue inhaler usage after the subsequent period.

Thus, the trained machine learning model can be used to predict future rescue inhaler usage based on comparing baseline and current/subsequent rescue inhaler usage, e.g. baseline and current/subsequent rescue inhaler usage in non-contiguous baseline and current periods respectively. Excessive rescue inhaler usage, for instance, may be indicative of worsening of the subject's respiratory disease. The capability to predict future rescue inhaler usage may correspondingly provide a warning of, for example, an impending exacerbation. A clinical assessment may, in at least some embodiments, not be required to train the machine learning model because the label data can comprise the measure of each of the training subjects' usage of the inhaler determined after the subsequent period.

Alternatively or additionally, the measure of the respective training subject's usage of the inhaler may comprise one or more parameters relating to airflow during an inhalation performed by the respective training subject using the inhaler. The one or more parameters relating to airflow may comprise, or consist of, at least one of a peak inhalation flow, an inhalation volume, a time to peak inhalation flow, and an inhalation duration, as previously described.

Alternatively or additionally, the assessment of the respiratory disease may comprise an approximation of a clinically determined indication of the status of the subject's respiratory disease, for example, a “moderate” or “severe” exacerbation as defined herein, as previously described.

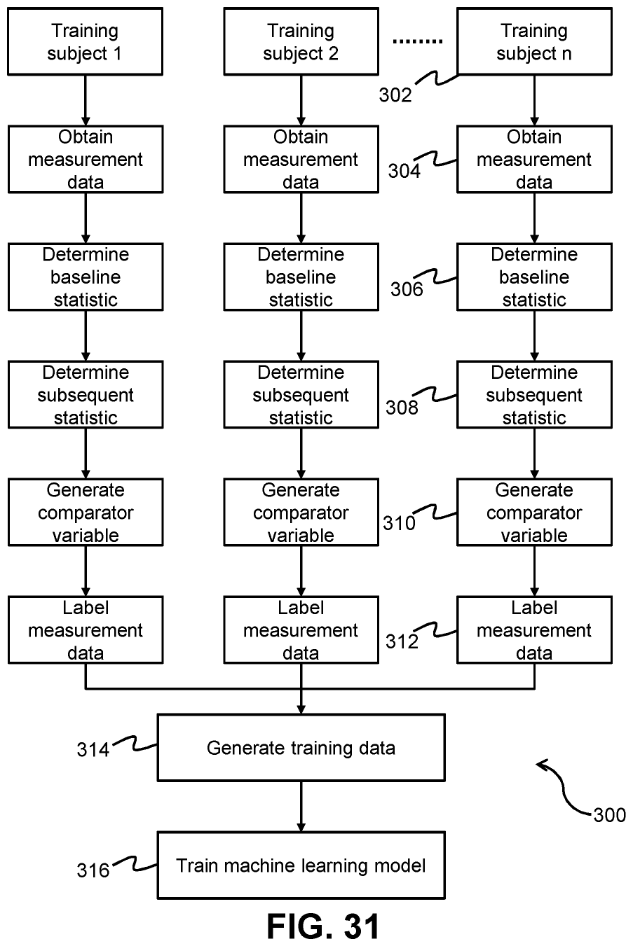

FIG. 31 shows a method 300 for training a machine learning model for use in generating an assessment of a respiratory disease in a subject according to another example. This method 300 comprises, for each of a plurality of training subjects 302, obtaining 304 measurement data comprising data relating to the training subject's usage of an inhaler. The inhaler is configured to deliver a rescue medicament to the training subject and has a use determination system configured to determine usage of the inhaler by the training subject, as previously described.

A baseline statistic relating to usage of the inhaler in a baseline period is determined at step 306. A subsequent statistic relating to usage of the inhaler in a subsequent period is also determined at 308. An intervening period may separate the subsequent period from the baseline period, similarly to in the above-described embodiments of the method for generating the assessment of the subject's respiratory disease in which the baseline period is separated from the current period by the intervening period.

A comparator variable is generated at step 310 for each of the plurality of training subjects. Generating 310 the comparator variable comprises comparing the subsequent statistic and the baseline statistic.

The method 300 further comprises labelling 312 the measurement data according to whether the comparator variable exceeds a predetermined threshold, generating 314 training data comprising the labelled measurement data, and training 316 the machine learning model using the labelled measurement data for the plurality of training subjects. The machine learning model is thereby trained to generate an assessment of the respiratory disease.

A clinical study was carried out in order to assess the factors influencing the probability of an asthma exacerbation. The following should be regarded as an explanatory and non-limiting example.

Albuterol administered using the ProAir Digihaler marketed by Teva Pharmaceutical Industries was utilized in this 12-week, open-label study, although the results of the study are more generally applicable to other rescue medicaments delivered using other device types.

Patients (≥18 years old) with exacerbation-prone/poorly controlled asthma were recruited to the study. The patients had an Asthma Control Questionnaire-5 (ACQ-5) score of ≥1.5; had experienced ≥1 episode of exacerbation in the 12 months prior to the study; had stable maintenance medicament dosing for at least 3 months prior to the study; and were taking a moderate dose ICS and/or another maintenance medicament.

Patients used the ProAir Digihaler (albuterol 90 mcg as the sulfate with a lactose carrier, 1-2 inhalations every 4 hours) as needed. The ProAir Digihaler replaced the patients' other rescue medications.

The electronics module of the ProAir Digihaler recorded each use, i.e. each inhalation, and parameters relating to airflow during each inhalation: peak inspiratory flow, volume inhaled, time to peak flow and inhalation duration. Data were downloaded from the inhalers and, together with clinical data, subjected to a machine-learning algorithm to develop models predictive of an impending exacerbation.

The diagnosis of a clinical asthma exacerbation (CAE) in this example was based on the American Thoracic Society/European Respiratory Society statement (H. K. Reddel et al., Am J Respir Crit Care Med. 2009, 180(1), 59-99). It includes both a “severe CAE” or a “moderate CAE.”

A severe CAE is defined as a CAE that involves worsening asthma that requires oral steroid (prednisone or equivalent) for at least three days and hospitalization. A moderate CAE requires oral steroid (prednisone or equivalent) for at least three days or hospitalization.

The objective and primary endpoint of the study was to explore the patterns and amount of albuterol use, as captured by the Digihaler, alone and in combination with other study data, such as the parameters relating to airflow during inhalation, physical activity, sleep, etc., preceding a CAE. This study represents the first successful attempt to develop a model to predict CAE derived from the use of a rescue medication inhaler device equipped with an integrated sensor and capable of measuring inhalation parameters.

It was found that 360 patients performed ≥1 valid inhalation from the Digihaler. These 360 patients were included in the analysis. Of these, 64 patients experienced a total of 78 CAEs. A total of 32970 rescue inhalations were recorded.

The mean age was 50.0 years, and 80.6% of the patients were female.

Over the full study period, mean (SD) peak inhalation flow and inhalation volume were 71.8 (23.2) L/min and 1.44 (0.77) L, respectively.

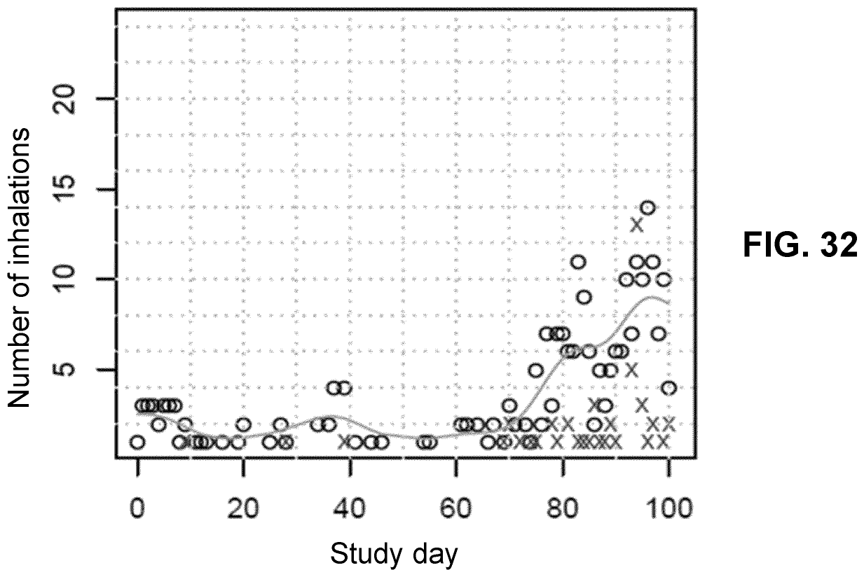

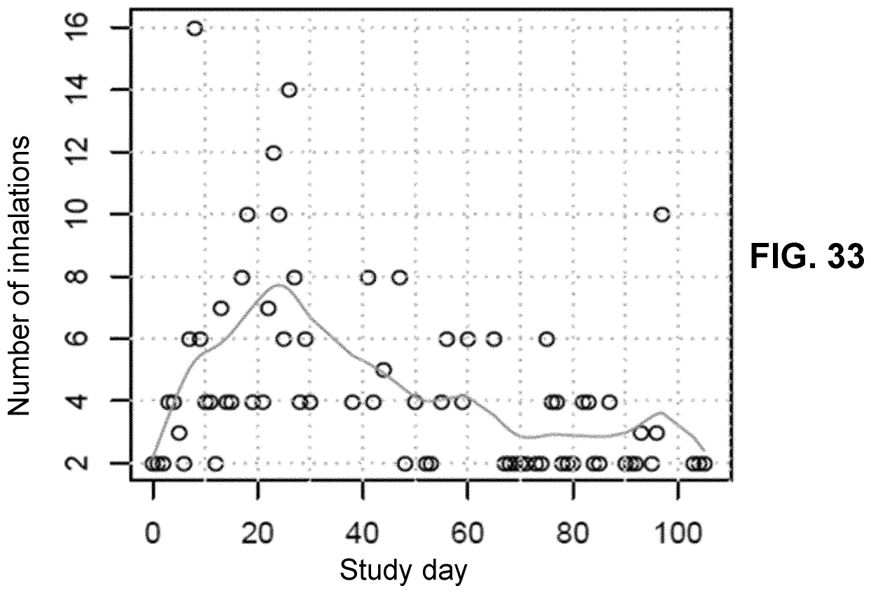

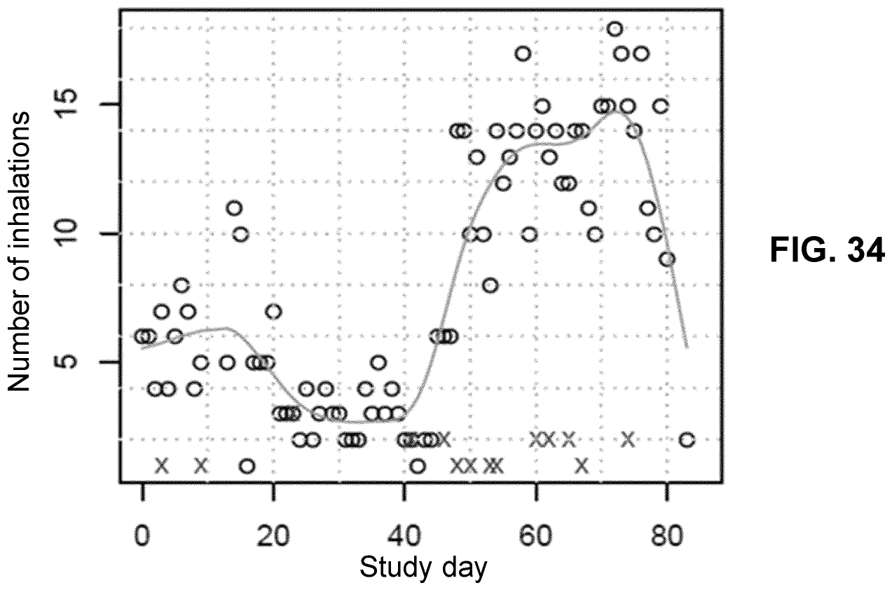

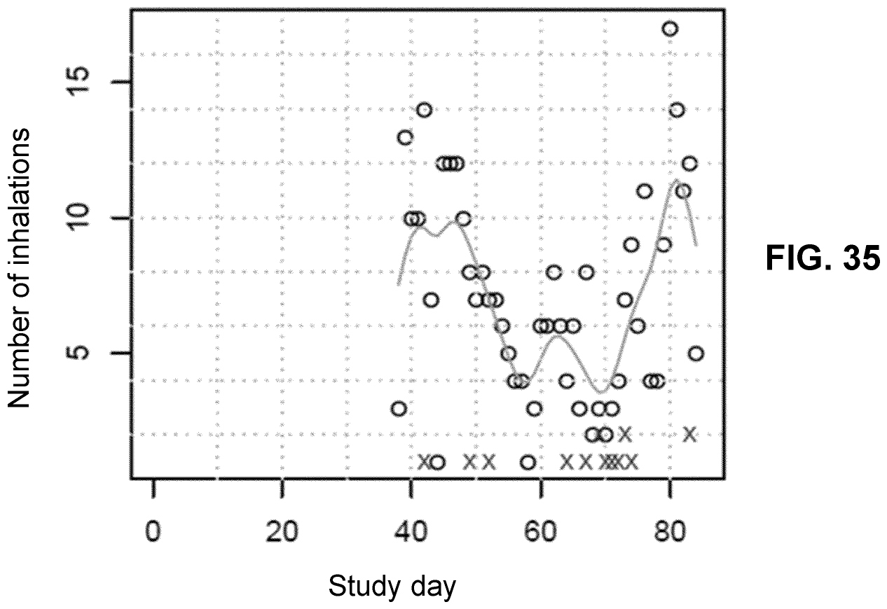

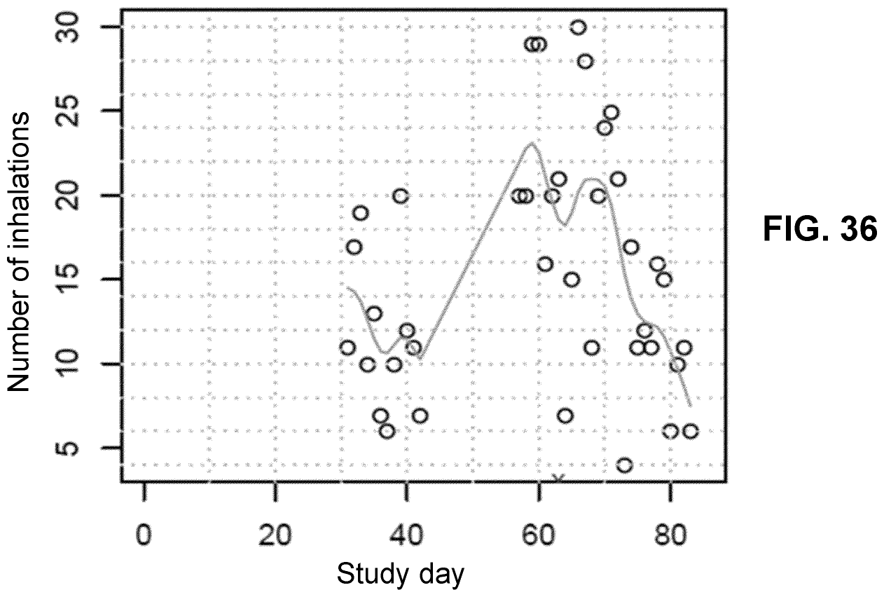

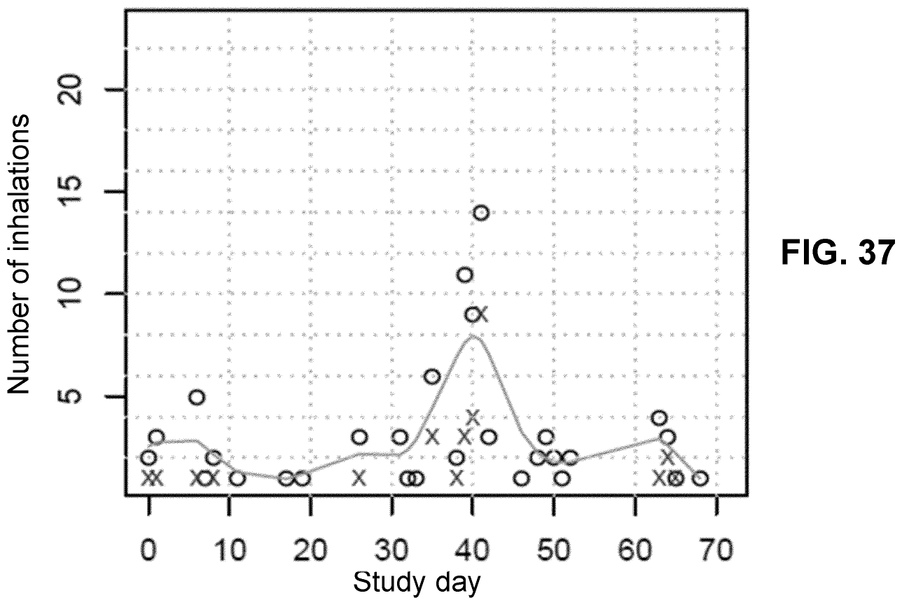

“SABA bursts” were used in this study as a surrogate for probable unconfirmed exacerbations. FIGS. 32 to 38 provide examples from the study of patients showing possible exacerbations which were not clinically diagnosed.

A “SABA burst” was defined using the results of this study:
a daily mean of at least three inhalations in the last two days; and
an increase in daily mean inhalations in the last two days compared with previous two weeks.

The increase in daily mean inhalations in the last two days compared with previous two weeks may be determined by:
daily average number of inhalations in last 2 days (current period) is a factor of 2 or more than the daily average number of inhalations in previous 2 weeks; and
daily average number of inhalations in last 2 days (current period) is +3 or more than daily average number of inhalations in previous 2 weeks.

Multiple SABA bursts within a 7-day period were counted as one burst. In other words, a series of SABA bursts with no more than 7 days between consecutive SABA burst were counted as one burst.

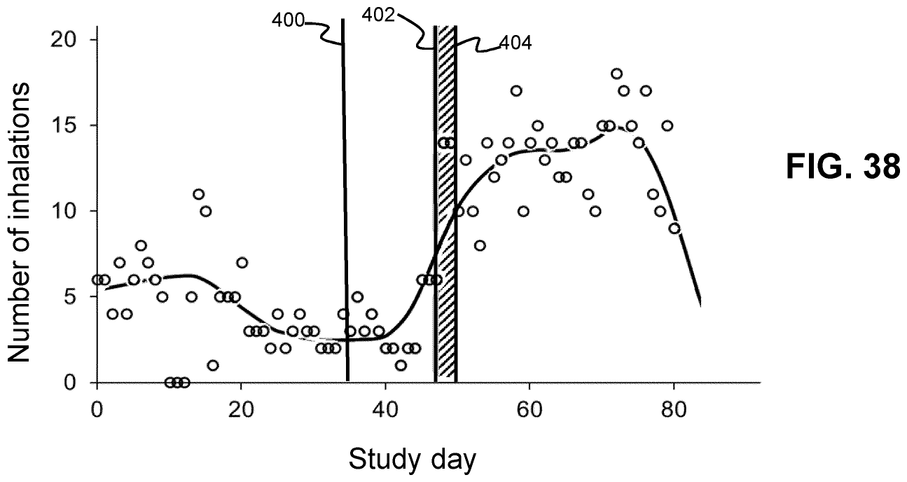

Referring to the patient example provided in FIG. 38 in the period which between days 34 to 47 (between the lines 400 and 402), the daily mean number of inhalations was 3.5. The daily mean number of inhalations between days 48 to 49 (between the lines 402 and 404) was 14. The increase in the daily mean number of inhalations is 10.5. This increase combined the absolute number of daily mean inhalations being ≥3 constitutes a SABA burst as defined for this study. Thus, an unconfirmed/undiagnosed exacerbation is considered likely to have taken place in this example.

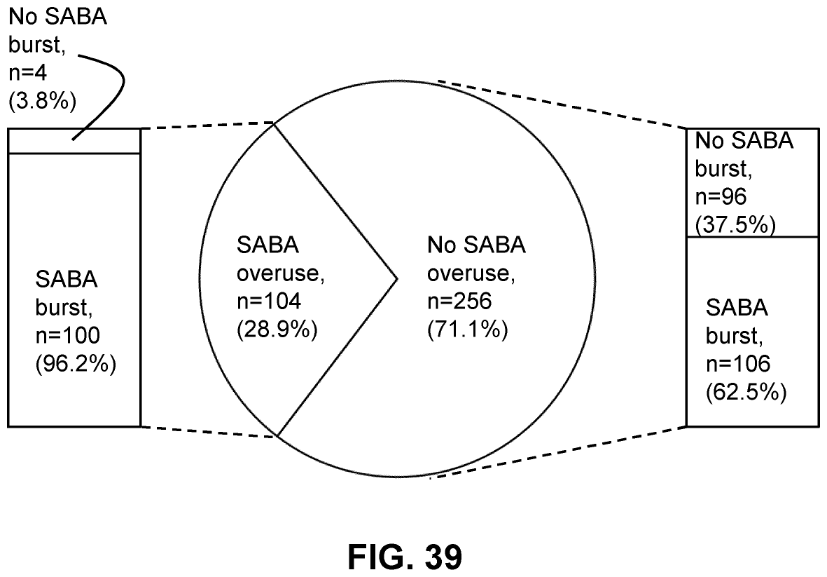

FIG. 39 provides a chart showing the rescue inhaler usage of a group of subjects. In particular, the chart shows that, of all patients, 28.9% showed continuous SABA overuse; a larger proportion of SABA overusers had SABA bursts (96.2%) compared with SABA non-overusers (62.5%). SABA overusers also had a larger mean number of SABA bursts per patient than SABA non-overusers (2.07 vs 1.02).

Thus, changes in patterns of rescue inhaler, and in particular short-acting beta-agonist (SABA) use, may indicate worsening of disease or the development of an exacerbation. Identifying and acting upon periods of increased rescue inhaler use may assist to halt the progression of exacerbations and improve clinical outcomes.

These data confirm the ability of the Digihaler to provide valuable information about patterns of SABA use, including continuous overuse, and inhalation parameters. Such information could uncover worsening disease control, or onset of a clinical exacerbation. A better understanding of these events may improve asthma management and improve clinical outcomes.

FIGS. 40 to 43 provide a non-limiting example of an inhaler 100 which may be included in the system 10.

FIG. 40 provides a front perspective view of an inhaler 100, according to a non-limiting example. The inhaler 100 may, for example, be a breath-actuated inhaler. The inhaler 100 may include a top cap 102, a main housing 104, a mouthpiece 106, a mouthpiece cover 108, an electronics module 120, and/or an air vent 126. The mouthpiece cover 108 may be hinged to the main housing 104 so that it may open and close to expose the mouthpiece 106. Although illustrated as a hinged connection, the mouthpiece cover 106 may be connected to the inhaler 100 through other types of connections. Moreover, while the electronics module 120 is illustrated as housed within the top cap 102 at the top of the main housing 104, the electronics module 120 may be integrated and/or housed within main body 104 of the inhaler 100.

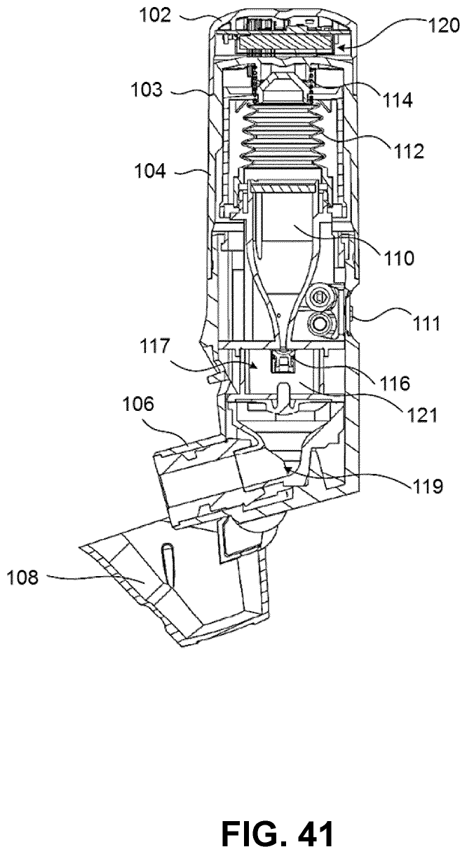

FIG. 41 provides a cross-sectional interior perspective view of the example inhaler 100. Inside the main housing 104, the inhaler 100 may include a medication reservoir 110 (e.g. a hopper), a bellows 112, a bellows spring 114, a yoke (not visible), a dosing cup 116, a dosing chamber 117, a deagglomerator 121, and a flow pathway 119. The medication reservoir 110 may include medication, such as dry powder medication, for delivery to the subject. When the mouthpiece cover 108 is moved from the closed to the open position, the bellows 112 may compress to deliver a dose of medication from the medication reservoir 110 to the dosing cup 116. Thereafter, a subject may inhale through the mouthpiece 106 in an effort to receive the dose of medication.

The airflow generated from the subject's inhalation may cause the deagglomerator 121 to aerosolize the dose of medication by breaking down the agglomerates of the medicament in the dose cup 116. The deagglomerator 121 may be configured to aerosolize the medication when the airflow through the flow pathway 119 meets or exceeds a particular rate, or is within a specific range. When aerosolized, the dose of medication may travel from the dosing cup 116, into the dosing chamber 117, through the flow pathway 119, and out of the mouthpiece 106 to the subject. If the airflow through the flow pathway 119 does not meet or exceed a particular rate, or is not within a specific range, the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, a single dose of medication may remain in the dosing cup until the dose has been aerosolized by the deagglomerator 121. When a dose of medication is delivered, a dose confirmation may be stored in memory at the inhaler 100 as dose confirmation information.

As the subject inhales through the mouthpiece 106, air may enter the air vent to provide a flow of air for delivery of the medication to the subject. The flow pathway 119 may extend from the dosing chamber 117 to the end of the mouthpiece 106, and include the dosing chamber 117 and the internal portions of the mouthpiece 106. The dosing cup 116 may reside within or adjacent to the dosing chamber 117. Further, the inhaler 100 may include a dose counter 111 that is configured to be initially set to a number of total doses of medication within the medication reservoir 110 and to decrease by one each time the mouthpiece cover 108 is moved from the closed position to the open position.

The top cap 102 may be attached to the main housing 104. For example, the top cap 102 may be attached to the main housing 104 through the use of one or more clips that engage recesses on the main housing 104. The top cap 102 may overlap a portion of the main housing 104 when connected, for example, such that a substantially pneumatic seal exists between the top cap 102 and the main housing 104.

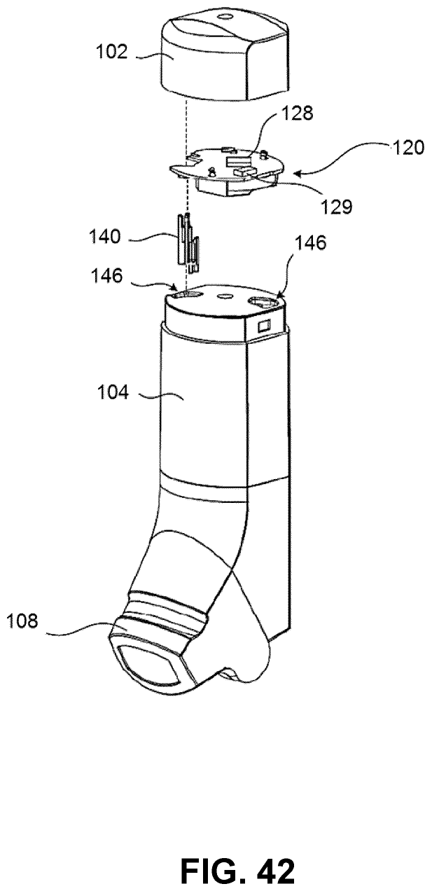

FIG. 42 is an exploded perspective view of the example inhaler 100 with the top cap 102 removed to expose the electronics module 120. As shown in FIG. 42, the top surface of the main housing 104 may include one or more (e.g. two) orifices 146. One of the orifices 146 may be configured to accept a slider 140. For example, when the top cap 102 is attached to the main housing 104, the slider 140 may protrude through the top surface of the main housing 104 via one of the orifices 146.

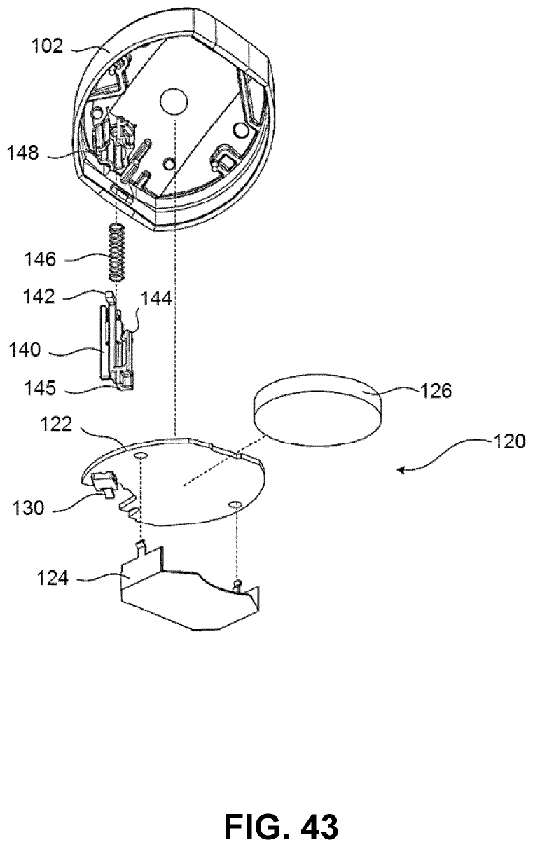

FIG. 43 is an exploded perspective view of the top cap 102 and the electronics module 120 of the example inhaler 100. As shown in FIG. 43, the slider 140 may define an arm 142, a stopper 144, and a distal end 145. The distal end 145 may be a bottom portion of the slider 140. The distal end 145 of the slider 140 may be configured to abut the yoke that resides within the main housing 104 (e.g. when the mouthpiece cover 108 is in the closed or partially open position). The distal end 145 may be configured to abut a top surface of the yoke when the yoke is in any radial orientation. For example, the top surface of the yoke may include a plurality of apertures (not shown), and the distal end 145 of the slider 140 may be configured to abut the top surface of the yoke, for example, whether or not one of the apertures is in alignment with the slider 140.

The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g. abut) an upper portion (e.g. a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke when the mouthpiece cover 108 is closed.

The distal end 145 of the slider 145 may also remain in contact with the yoke while the mouthpiece cover 108 is being opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g. axial) travel of the slider 140. This limit may be less than the vertical travel of the yoke. Thus, as the mouthpiece cover 108 is moved to a fully open position, the yoke may continue to move in a vertical direction towards the mouthpiece 106 but the stopper 144 may stop the vertical travel of the slider 140 such that the distal end 145 of the slider 140 may no longer be in contact with the yoke.

More generally, the yoke may be mechanically connected to the mouthpiece cover 108 and configured to move to compress the bellows spring 114 as the mouthpiece cover 108 is opened from the closed position and then release the compressed bellows spring 114 when the mouthpiece cover reaches the fully open position, thereby causing the bellows 112 to deliver the dose from the medication reservoir 110 to the dosing cup 116. The yoke may be in contact with the slider 140 when the mouthpiece cover 108 is in the closed position. The slider 140 may be arranged to be moved by the yoke as the mouthpiece cover 108 is opened from the closed position and separated from the yoke when the mouthpiece cover 108 reaches the fully open position. This arrangement may be regarded as a non-limiting example of the previously described dose metering assembly, since opening the mouthpiece cover 108 causes the metering of the dose of the medicament.

The movement of the slider 140 during the dose metering may cause the slider 140 to engage and actuate a switch 130. The switch 130 may trigger the electronics module 120 to register the dose metering. The slider 140 and switch 130 together with the electronics module 120 may thus correspond to a non-limiting example of the use determination system 12B described above. The slider 140 may be regarded in this example as the means by which the use determination system 12B is configured to register the metering of the dose by the dose metering assembly, each metering being thereby indicative of the inhalation performed by the subject using the inhaler 100.

Actuation of the switch 130 by the slider 140 may also, for example, cause the electronics module 120 to transition from the first power state to a second power state, and to sense an inhalation by the subject from the mouthpiece 106.

The electronics module 120 may include a printed circuit board (PCB) assembly 122, a switch 130, a power supply (e.g. a battery 126), and/or a battery holder 124. The PCB assembly 122 may include surface mounted components, such as a sensor system 128, a wireless communication circuit 129, the switch 130, and or one or more indicators (not shown), such as one or more light emitting diodes (LEDs). The electronics module 120 may include a controller (e.g. a processor) and/or memory. The controller and/or memory may be physically distinct components of the PCB 122. Alternatively, the controller and memory may be part of another chipset mounted on the PCB 122, for example, the wireless communication circuit 129 may include the controller and/or memory for the electronics module 120. The controller of the electronics module 120 may include a microcontroller, a programmable logic device (PLD), a microprocessor, an application specific integrated circuit (ASIC), a field programmable gate array (FPGA), or any suitable processing device or control circuit.

The controller may access information from, and store data in the memory. The memory may include any type of suitable memory, such as non-removable memory and/or removable memory. The non-removable memory may include random-access memory (RAM), read-only memory (ROM), or any other type of memory storage device. The removable memory may include a subscriber identity module (SIM) card, a memory stick, a secure digital (SD) memory card, and the like. The memory may be internal to the controller. The controller may also access data from, and store data in, memory that is not physically located within the electronics module 120, such as on a server or a smart phone.

The sensor system 128 may include one or more sensors. The sensor system 128 may be an example of the sensor system 12A. The sensor system 128 may include one or more sensors, for example, of different types, such as, but not limited to one or more pressure sensors, temperature sensors, humidity sensors, orientation sensors, acoustic sensors, and/or optical sensors. The one or more pressure sensors may include a barometric pressure sensor (e.g. an atmospheric pressure sensor), a differential pressure sensor, an absolute pressure sensor, and/or the like. The sensors may employ microelectromechanical systems (MEMS) and/or nanoelectromechanical systems (NEMS) technology.

The sensor system 128 may be configured to provide an instantaneous reading (e.g. pressure reading) to the controller of the electronics module 120 and/or aggregated readings (e.g. pressure readings) over time. As illustrated in FIGS. 41 and 42, the sensor system 128 may reside outside the flow pathway 119 of the inhaler 100, but may be pneumatically coupled to the flow pathway 119.

The controller of the electronics module 120 may receive signals corresponding to measurements from the sensor system 128. The controller may calculate or determine one or more airflow metrics using the signals received from the sensor system 128. The airflow metrics may be indicative of a profile of airflow through the flow pathway 119 of the inhaler 100. For example, if the sensor system 128 records a change in pressure of 0.3 kilopascals (kPa), the electronics module 120 may determine that the change corresponds to an airflow rate of approximately 45 liters per minute (Lpm) through the flow pathway 119.

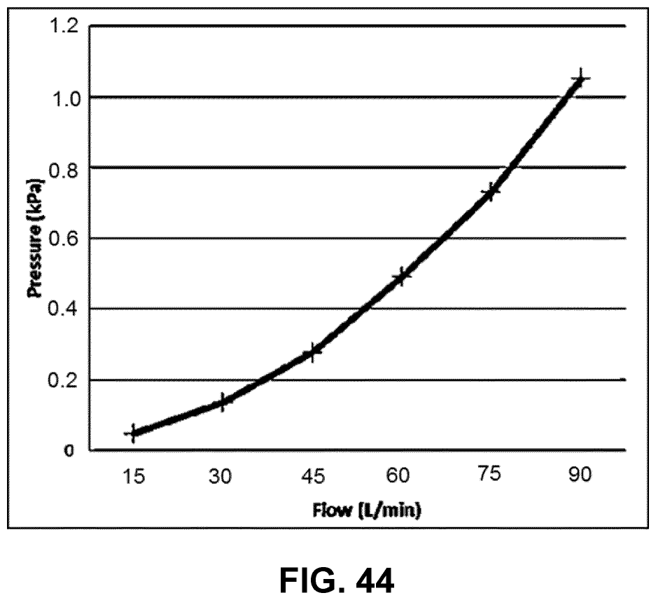

FIG. 44 shows a graph of airflow rates versus pressure. The airflow rates and profile shown in FIG. 44 are merely examples and the determined rates may depend on the size, shape, and design of the inhalation device 100 and its components.

The one or more processors 14 may generate personalized data in real-time by comparing signals received from the sensor system 128 and/or the determined airflow metrics to one or more thresholds or ranges, for example, as part of an assessment of how the inhaler 100 is being used and/or whether the use is likely to result in the delivery of a full dose of medication. For example, where the determined airflow metric corresponds to an inhalation with an airflow rate below a particular threshold, the one or more processors 14 may determine that there has been no inhalation or an insufficient inhalation from the mouthpiece 106 of the inhaler 100. If the determined airflow metric corresponds to an inhalation with an airflow rate above a particular threshold, the one or more processors 14 may determine that there has been an excessive inhalation from the mouthpiece 106. If the determined airflow metric corresponds to an inhalation with an airflow rate within a particular range, the one or more processors 14 may determine that the inhalation is “good”, or likely to result in a full dose of medication being delivered.

The pressure measurement readings and/or the computed airflow metrics may be indicative of the quality or strength of inhalation from the inhaler 100. For example, when compared to a particular threshold or range of values, the readings and/or metrics may be used to categorize the inhalation as a certain type of event, such as a good inhalation event, a low inhalation event, a no inhalation event, or an excessive inhalation event. The categorization of the inhalation may be usage parameters stored as personalized data of the subject.

The no inhalation event may be associated with pressure measurement readings and/or airflow metrics below a particular threshold, such as an airflow rate less than 30 Lpm. The no inhalation event may occur when a subject does not inhale from the mouthpiece 106 after opening the mouthpiece cover 108 and during the measurement cycle. The no inhalation event may also occur when the subject's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

The low inhalation event may be associated with pressure measurement readings and/or airflow metrics within a particular range, such as an airflow rate between 30 Lpm and 45 Lpm. The low inhalation event may occur when the subject inhales from the mouthpiece 106 after opening the mouthpiece cover 108 and the subject's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, such as an airflow rate between 45 Lpm and 200 Lpm. The good inhalation event may occur when the subject inhales from the mouthpiece 106 after opening the mouthpiece cover 108 and the subject's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

The excessive inhalation event may be associated with pressure measurement readings and/or airflow metrics above the good inhalation event, such as an airflow rate above 200 Lpm. The excessive inhalation event may occur when the subject's inspiratory effort exceeds the normal operational parameters of the inhaler 100. The excessive inhalation event may also occur if the device 100 is not properly positioned or held during use, even if the subject's inspiratory effort is within a normal range. For example, the computed airflow rate may exceed 200 Lpm if the air vent is blocked or obstructed (e.g. by a finger or thumb) while the subject is inhaling from the mouthpiece 106.

Any suitable thresholds or ranges may be used to categorize a particular event. Some or all of the events may be used. For example, the no inhalation event may be associated with an airflow rate below 45 Lpm and the good inhalation event may be associated with an airflow rate between 45 Lpm and 200 Lpm. As such, the low inhalation event may not be used at all in some cases.

The pressure measurement readings and/or the computed airflow metrics may also be indicative of the direction of flow through the flow pathway 119 of the inhaler 100. For example, if the pressure measurement readings reflect a negative change in pressure, the readings may be indicative of air flowing out of the mouthpiece 106 via the flow pathway 119. If the pressure measurement readings reflect a positive change in pressure, the readings may be indicative of air flowing into the mouthpiece 106 via the flow pathway 119. Accordingly, the pressure measurement readings and/or airflow metrics may be used to determine whether a subject is exhaling into the mouthpiece 106, which may signal that the subject is not using the device 100 properly.

The personalized data collected from, or calculated based on, the usage of the inhaler 100 (e.g. pressure metrics, airflow metrics, lung function metrics, dose confirmation information, etc.) may be computed and/or assessed via external devices as well (e.g. partially or entirely). More specifically, the wireless communication circuit 129 in the electronics module 120 may include a transmitter and/or receiver (e.g. a transceiver), as well as additional circuitry. For example, the wireless communication circuit 129 may include a Bluetooth chip set (e.g. a Bluetooth Low Energy chip set), a ZigBee chipset, a Thread chipset, etc. As such, the electronics module 120 may wirelessly provide the personalized data, such as pressure measurements, airflow metrics, lung function metrics, dose confirmation information, and/or other conditions related to usage of the inhaler 100, to an external device, including a smart phone. The personalized data may be provided in real time to the external device to enable the above-described assessment generation based on real-time data from the inhaler 100 that indicates time of use, how the inhaler 100 is being used, and personalized data about the user of the inhaler, such as real-time data related to the subject's lung function and/or medical treatment. The external device may include software for processing the received information and for providing compliance and adherence feedback to users of the inhaler 100 via a graphical user interface (GUI).

The airflow metrics may include personalized data that is collected from the inhaler 100 in real-time, such as one or more of an average flow of an inhalation/exhalation, a peak flow of an inhalation/exhalation (e.g. a maximum inhalation received), a volume of an inhalation/exhalation, a time to peak of an inhalation/exhalation, and/or the duration of an inhalation/exhalation. The airflow metrics may also be indicative of the direction of flow through the flow pathway 119. That is, a negative change in pressure may correspond to an inhalation from the mouthpiece 106, while a positive change in pressure may correspond to an exhalation into the mouthpiece 106. When calculating the airflow metrics, the electronics module 120 may be configured to eliminate or minimize any distortions caused by environmental conditions. For example, the electronics module 120 may re-zero to account for changes in atmospheric pressure before or after calculating the airflow metrics. The one or more pressure measurements and/or airflow metrics may be timestamped and stored in the memory of the electronics module 120.

In addition to the airflow metrics, the inhaler 100, or another computing device, may use the airflow metrics to generate additional personalized data. For example, the controller of the electronics module 120 of the inhaler 100 may translate the airflow metrics into other metrics that indicate the subject's lung function and/or lung health that are understood to medical practitioners, such as peak inspiratory flow metrics, peak expiratory flow metrics, and/or forced expiratory volume in 1 second (FEV1), for example. The electronics module 120 of the inhaler may determine a measure of the subject's lung function and/or lung health using a mathematical model such as a regression model. The mathematical model may identify a correlation between the total volume of an inhalation and FEV1. The mathematical model may identify a correlation between peak inspiratory flow and FEV1. The mathematical model may identify a correlation between the total volume of an inhalation and peak expiratory flow. The mathematical model may identify a correlation between peak inspiratory flow and peak expiratory flow.

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a particular battery capacity that may affect the life of the battery 126.

As will be further discussed below, the distribution of power from the battery 126 to the one or more components of the PCB 122 may be managed to ensure the battery 126 can power the electronics module 120 over the useful life of the inhaler 100 and/or the medication contained therein.

In a connected state, the communication circuit and memory may be powered on and the electronics module 120 may be “paired” with an external device, such as a smart phone. The controller may retrieve data from the memory and wirelessly transmit the data to the external device. The controller may retrieve and transmit the data currently stored in the memory. The controller may also retrieve and transmit a portion of the data currently stored in the memory. For example, the controller may be able to determine which portions have already been transmitted to the external device and then transmit the portion(s) that have not been previously transmitted. Alternatively, the external device may request specific data from the controller, such as any data that has been collected by the electronics module 120 after a particular time or after the last transmission to the external device. The controller may retrieve the specific data, if any, from the memory and transmit the specific data to the external device.

The data stored in the memory of the electronics module 120 (e.g. the signals generated by the switch 130, the pressure measurement readings taken by the sensory system 128 and/or the airflow metrics computed by the controller of the PCB 122) may be transmitted to an external device, which may process and analyze the data to determine the usage parameters associated with the inhaler 100. Further, a mobile application residing on the mobile device may generate feedback for the user based on data received from the electronics module 120. For example, the mobile application may generate daily, weekly, or monthly report, provide confirmation of error events or notifications, provide instructive feedback to the subject, and/or the like.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

What is claimed is:

1.-5. (canceled)

6. The method according to claim 59, wherein the assessment of the respiratory disease comprises an approximation to a clinically determined indication of the status of the subject's respiratory disease.

7. The method according to claim 59, wherein the assessment of the respiratory disease comprises a prediction of the subject later suffering an exacerbation of their respiratory disease.

8.-13. (canceled)

14. The method according to claim 59, comprising controlling a user interface to communicate a notification based on the generated assessment of the respiratory disease.

15. The method according to claim 59, wherein the baseline statistic comprises one or more of a baseline average number of rescue inhalations using the inhaler per unit time, a baseline standard deviation of the number of rescue inhalations using the inhaler per unit time, or a baseline coefficient of variance of the number of rescue inhalations per unit time, calculated over the baseline period.

16. The method according to claim 15, wherein the baseline average number of rescue inhalations per unit time comprises a mean, a median, or a mode of the number of rescue inhalations using the inhaler per unit time calculated over the baseline period.

17. (canceled)

18. (canceled)

19. The method according to claim 59, wherein the duration of the baseline period is 10 to 30 days.

20. (canceled)

21. The method according to claim 59, wherein the subject did not experience an exacerbation of their respiratory disease in the baseline period.

22.-28. (canceled)

29. The method according to claim 59 any of claims 1 to 28, wherein the duration of the intervening period is 3 to 15 days.

30. (canceled)

31. The method according to claim 59, wherein determining the current statistic comprises determining a total current number of rescue inhalations summed over the current period.

32. The method according to claim 59, wherein the current statistic comprises one or more of a current average number of rescue inhalations using the inhaler per unit time, a current standard deviation of the number of rescue inhalations using the inhaler per unit time, or a current coefficient of variance of the number of rescue inhalations per unit time, calculated over the current period.

33. (canceled)

34. The method according to claim 32, wherein the baseline statistic comprises a baseline average number of rescue inhalations using the inhaler per unit time, calculated over the baseline period, wherein said comparing the current statistic and the baseline statistic comprises comparing the baseline average and the current average.

35. (canceled)

36. (canceled)

37. The method according to claim 34, wherein said comparing the current statistic and the baseline statistic comprises calculating a ratio of the current average to the baseline average.

38. (canceled)

39. (canceled)

40. The method according to claim 59, wherein the duration of the current period is 24 hours to 120 hours.

41.-44. (canceled)

45. The method according to claim 59, comprising:
generating the assessment of the respiratory disease in the subject as an output of a trained machine learning model;
determining a current inhalation parameter statistic from a determined parameter relating to airflow during an inhalation performed by the subject using an inhaler during the current period; and
applying the current inhalation parameter statistic or data derived from the current inhalation parameter statistic as an input or inputs to the trained machine learning model.

46.-49. (canceled)

50. The method according to claim 45, wherein the parameter relating to airflow is at least one of a peak inhalation flow, an inhalation volume, a time to peak inhalation flow, or an inhalation duration.

51. The method according to claim 59, wherein the rescue medicament is selected from albuterol, formoterol, budesonide combined with formoterol, beclomethasone combined with albuterol, and fluticasone combined with albuterol.

52.-55. (canceled)

56. The method according to claim 60, wherein the assessment of the respiratory disease comprises a clinically determined indication of the status of the subject's respiratory disease; optionally wherein the clinically determined indication is of an exacerbation of the respective subject's respiratory disease determined after the subsequent period.

57. (canceled)

58. (canceled)

59. A method for generating an assessment of a respiratory disease in a subject at a current point in time, the method comprising:
determining a baseline statistic relating to usage of an inhaler in a baseline period, wherein the inhaler is configured to deliver a rescue medicament to the subject, and has a use determination system configured to determine usage of the inhaler by the subject;
determining a current statistic relating to usage of the inhaler in a current period containing the current point in time, wherein an intervening period separates the current period from the baseline period;
generating a comparator variable, comprising comparing the current statistic and the baseline statistic; and
generating the assessment based on the comparator variable.

60. A method for training a machine learning model for use in generating an assessment of a respiratory disease in a subject, the method comprising:
for each of a plurality of training subjects,
obtaining measurement data, comprising data relating to the training subject's usage of an inhaler, wherein the inhaler is configured to deliver a rescue medicament to the training subject and has a use determination system configured to determine usage of the inhaler by the training subject;
determining a baseline statistic relating to usage of the inhaler in a baseline period;
determining a subsequent statistic relating to usage of the inhaler in a subsequent period, wherein an intervening period separates the subsequent period from the baseline period;
generating a comparator variable, comprising comparing the subsequent statistic and the baseline statistic; and
labelling the measurement data according to whether the comparator variable exceeds a predetermined threshold; and
training the machine learning model using the labelled measurement data for the plurality of training subjects, wherein the machine learning model is thereby trained to generate an assessment of the respiratory disease.

61. (canceled)

62. One or more non-transitory computer readable media having a computer program stored thereon, the computer program comprising computer program code which is configured, when said computer program is run on one or more physical computing devices, to cause said one or more physical computing devices to implement the method of claim 60.

63. (canceled)

64. (canceled)
